(19) United States
(12) Patent Application Publication
(10) Pub. No.: US 2023/0258648 A1
(43) Pub. Date: Aug. 17, 2023

(54) MARKERS FOR PREDICTING POSSIBILITIES OF SUBJECTS WITH DIABETES AND USE THEREOF

(71) Applicants: JIANGSU QLIFE MEDICAL TECHNOLOGY GROUP CO., LTD., Nanjing (CN); NANJING QLIFE MEDICAL TECHNOLOGY CO., LTD., Nanjing (CN)

(72) Inventors: Xiaoliang CHENG, Nanjing (CN); Meijuan LI, Nanjing (CN); Yue ZHOU, Nanjing (CN); Wei ZHANG, Nanjing (CN); Kejia ZHENG, Nanjing (CN)

(73) Assignees: JIANGSU QLIFE MEDICAL TECHNOLOGY GROUP CO., LTD., Nanjing (CN); NANJING QLIFE MEDICAL TECHNOLOGY CO., LTD., Nanjing (CN)

(21) Appl. No.: 18/301,249

(22) Filed: Apr. 16, 2023

Related U.S. Application Data
(63) Continuation of application No. PCT/CN2021/134625, filed on Nov. 30, 2021.

(51) Int. Cl. G01N 33/68; G16H 50/30

(52) U.S. Cl. CPC G01N 33/6815; G16H 50/30; G01N 2800/042; G01N 2800/50

(57) ABSTRACT

The present disclosure provides a marker and use thereof in predicting a possibility of a subject with diabetes. The marker described may include at least one of α-hydroxybutyric acid (α-HB), 1,5-anhydroglucitol (1,5-AG), asymmetric dimethylarginine (ADMA), cystine, ethanolamine, taurine, L-leucine, L-tryptophan, hydroxylysine, and L-aspartate. The possibility of the subject with diabetes may be predicted using a prediction model (e.g., prediction models 2-5) related to the marker based on a concentration of the marker. The prediction model 2 is related to α-HB. The prediction model 3 is related to 1,5-AG and ADMA. The prediction model 4 is related to cystine, ethanolamine, taurine, L-leucine, L-tryptophan and hydroxylysine. The prediction model 5 is related to α-HB, 1,5-AG, cystine, ethanolamine, taurine and L-aspartate.

### CROSS-REFERENCE TO RELATED APPLICATION

This application is a Continuation of International Patent Application No. PCT/CN2021/134625, filed on Nov. 30, 2021, the contents of which are entirely incorporated herein by reference.

### TECHNICAL FIELD

The present disclosure relates to the field of diabetes detection, and in particular to a marker for predicting a possibility of a subject with diabetes and the use thereof.

### BACKGROUND

Diabetes is one of the four major non-communicable diseases in the world, and the number of patients with the disease has gradually increased in recent years. Currently, for gestational diabetes, the oral glucose tolerance test (OGTT) is the main method for early screening of diabetes, but the method has some drawbacks. For example, the OGTT requires a person for an overnight fast of at least 8 hours and consumption of a liquid containing 75 grams of glucose over 5 minutes, but some people (e.g., a pregnant woman) cannot easily apply the overnight fast, have difficulty in tolerating glucose drinks, and may have adverse reactions, e.g., nausea, vomiting, bloating, and headache. In addition, people with normal test results have had to undergo the OGTT, but have not any clinical benefit. Therefore, given the shortcomings of current method for detecting diabetes, it is desirable to provide a more objective, convenient, and non-adverse diabetes detection method.

### SUMMARY

According to an aspect of the present disclosure, there is provided a use of a marker in preparing a reagent, composition or kit for predicting a possibility of a subject with diabetes. The prediction may include: determining, based on a sample from the subject, a concentration of the marker, wherein the marker includes at least one of α-hydroxybutyric acid (α-HB), 1,5-anhydroglucitol (1,5-AG), asymmetric dimethylarginine (ADMA), cystine, ethanolamine, taurine, L-leucine, L-tryptophan, hydroxylysine, L-aspartic acid; and predicting, based on the concentration of the marker, the possibility of the subject with diabetes by using a prediction model related to the marker.

In some embodiments, the diabetes may include type 1 diabetes, type 2 diabetes, or gestational diabetes mellitus (GDM).

In some embodiments, the marker may include α-HB.

In some embodiments, the marker may include 1,5-AG and ADMA.

In some embodiments, the marker may include cystine, ethanolamine, taurine, L-leucine, L-tryptophan, and hydroxylysine.

In some embodiments, the marker may include α-HB, 1,5-AG, cystine, ethanolamine, taurine, and L-aspartic acid.

In some embodiments, the predicting, based on the concentration of the marker, the possibility of the subject with diabetes by using a prediction model related to the marker may include: outputting a prediction value from the prediction model by using the concentration of the marker as an input to the prediction model; and predicting the possibility of the subject having diabetes by comparing the prediction value to a threshold.

In some embodiments, the predicting the possibility of the subject having diabetes by comparing the prediction value to a threshold may include: predicting that the possibility of the subject with diabetes is high if the prediction value is greater than or equal to the threshold; or predicting that the possibility of the subject with diabetes is low if the prediction value is less than the threshold.

In some embodiments, the prediction model may be further related to an age and BMI of the subject.

$$log ( \frac{p}{1 - p} ) = - 13.38647 + 1.49950 * ( α - H B ) + 0.0 \; 7665 * age + 0.11713 * BMI$$

In some embodiments, the prediction model is represented by the equation of

where p represents a probability value of the subject with diabetes,

$$log ( \frac{p}{1 - p} )$$

represents an odds ratio, and α-HB represents a concentration of α-HB in μmol/L.

$$log ( \frac{p}{1 - p} ) = - 3.56131 + ( - 0.74606 ) * ( 1 , 5 - AG ) + ( - 1.40508 ) * A D M A + 0.0 \; 7688 * age + 0.12063 * BMI$$

In some embodiments, the prediction model is represented by the equation of
where p represents a probability value of the subject with diabetes,

$$log ( \frac{p}{1 - p} )$$

represents an odds ratio, and 1,5-anhydroglucitol and ADMA represent a concentration of 1,5-AG and ADMA, respectively, in μmol/L.

$$log ( \frac{p}{1 - p} ) = - 6.98386 + 1.5 \; 6579 * cystine + ( - 5.25949 ) * ethanolamine + 1.64365 * ( L - leucine ) + ( - 1.80619 ) * ( L - tryptophan ) + 0.7315 * hydroxylysine + 2.47105 * taurine + 0.08815 * age + 0.12894 * BMI$$

the prediction model is represented by the equation of

where p represents a probability value of the subject with diabetes,

$$log ( \frac{p}{1 - p} )$$

represents an odds ratio, and cystine, ethanolamine, L-leucine, L-tryptophan, hydroxylysine, and taurine represent concentrations of cystine, ethanolamine, L-leucine, L-tryptophan, hydroxylysine, and taurine, respectively, in μmol/L.

$$log ( \frac{p}{1 - p} ) = - 6.33027 + ( - 0.81716 ) * ( 1 , 5 - AG ) + 1.43266 * ( α - H B ) + 1.51073 * taurine + 0.9601 * ( L - aspartic acid ) + 1.26682 * cystine + ( - 5.1819 ) * ethanolamine + 0.0787 * age + 0.127 * BMI$$

the prediction model is represented by the equation of

where p represents a probability value of the subject with diabetes,

$$log ( \frac{p}{1 - p} )$$

represents a odds ratio, 1,5-AG, α-HB, taurine, L-aspartic acid, cystine and ethanolamine represent concentrations of 1,5-AG, α-HB, taurine, L-aspartic acid, cystine and ethanolamine in μmol/L.

In some embodiments, all AUC values of the prediction model are greater than 0.7 in a validation set and a sensitivity and a specificity of the prediction model are greater than 65% in the validation set.

According to another aspect of the present disclosure, there is also provided a marker for predicting a possibility of a subject with diabetes, wherein the marker comprises α-HB, 1,5-AG, ADMA, cystine, ethanolamine, taurine, L-leucine, L-tryptophan, hydroxylysine, and L-aspartic acid.

According to a further aspect of the present disclosure, there is also provided a use of a prediction model in preparing a reagent, composition, or kit for predicting a possibility of a subject with diabetes. The prediction model is related to a marker for predicting the possibility of the subject with diabetes, wherein the marker includes at least one of α-HB, 1,5-AG, ADMA, cystine, ethanolamine, taurine, L-leucine, L-tryptophan, hydroxylysine, L-aspartic acid; an input of the prediction model is a concentration of the marker and an output of the prediction model is a prediction value, the prediction value is compared with a threshold to predict the possibility of the subject with diabetes.

According to a further aspect of the present disclosure, there is provided a method for treating diabetes. The method may comprise: determining, based on a sample from a subject, a concentration of a marker, wherein the marker includes at least one of α-HB, 1,5-AG, ADMA, cystine, ethanolamine, taurine, L-leucine, L-tryptophan, hydroxylysine, and L-aspartic acid; predicting a possibility of the subject with diabetes by using a prediction model related to the marker based on the concentration of the marker; and if a prediction result is that the subject has diabetes, administering to the subject a drug for treating diabetes.

According to a further aspect of the present disclosure, there is provided a system for predicting a possibility of a subject with diabetes. The system may comprise an acquisition module used to obtain a concentration of a marker in a sample of the subject, wherein the marker includes at least one of α-HB, 1,5-AG, ADMA, cystine, ethanolamine, taurine, L-leucine, L-tryptophan, hydroxylysine, and L-aspartic acid; a training module used to obtain a prediction model by training an initial model using a training set, the prediction model being related to the marker; and a prediction module used to predict the possibility of the subject with diabetes by using the prediction model based on the concentration of the marker.

### BRIEF DESCRIPTION OF THE DRAWINGS

The present disclosure is further described by way of exemplary embodiments, which are described in detail by way of the accompanying drawings. These embodiments are not limiting, and in these embodiments the same numbering indicates the same structure wherein:

FIG. 1A and FIG. 1B illustrate total ion flow chromatograms of 25 amino acids and their derivatives in standards and a plasma sample, respectively, according to some embodiments of the present disclosure;

FIG. 2A and FIG. 2B illustrate total ion flow chromatograms of 1,5-AG, TMAO, ADMA and SDMA in standards and a plasma sample, respectively, according to some embodiments of the present disclosure;

FIG. 3A and FIG. 3B illustrate total ion flow chromatograms of α-HB, OA and LGPC in standards and a plasma sample, respectively, according to some embodiments of the present disclosure;

FIGS. 4A to 4L illustrate distribution diagrams of the significant relationships of all variables of five prediction models and GDM according to some embodiments of the present disclosure, where black indicates GDM and white indicates non-GDM; and

FIGS. 5A to 5J illustrate ROC curves of five prediction models in a training set and a validation set according to some embodiments of the present disclosure.

### DETAILED DESCRIPTION

The technical schemes of embodiments of the present disclosure will be more clearly described below, and the accompanying drawings need to be configured in the description of the embodiments will be briefly described below. Obviously, the drawings in the following description are merely some examples or embodiments of the present disclosure, and will be applied to other similar scenarios according to these accompanying drawings without paying creative labor. Unless obviously obtained from the context or the context illustrates otherwise, the same numeral in the drawings refers to the same structure or operation.

It should be understood that the “system”, “device”, “unit” and/or “module” used herein is a method for distinguishing different components, elements, portions, parts or assemblies of different levels. However, if other words may achieve the same purpose, the words may be replaced by other expressions.

As shown in the present disclosure and claims, unless the context clearly prompts the exception, “a”, “an”, “one”, and/or “the” is not specifically singular form, and the plural form may be included. It will be further understood that the terms “comprise,” “comprises,” and/or “comprising,” “include,” “includes,” and/or “including,” when used in present disclosure, specify the presence of stated features, integers, steps, operations, elements, and/or components, but do not preclude the presence or addition of one or more other features, integers, steps, operations, elements, components, and/or groups thereof.

The flowcharts are used in present disclosure to illustrate the operations performed by the system according to the embodiment of the present disclosure. It should be understood that the preceding or following operations is not necessarily performed in order to accurately. Instead, the operations may be processed in reverse order or simultaneously. Moreover, one or more other operations may be added to the flowcharts. One or more operations may be removed from the flowcharts.

The present disclosure provides a marker for predicting a possibility of a subject with diabetes, further provides a use of a marker in preparing a reagent, composition, or kit for predicting a possibility of a subject with diabetes, further provides a use of a prediction model in preparing a reagent, composition, or kit for predicting a possibility of a subject with diabetes, further provides a method for treating diabetes, and further provides a system for predicting a possibility of a subject with diabetes. In the present disclosure, the marker may include at least one of α-HB, 1,5-AG, ADMA, cystine, ethanolamine, taurine, L-leucine, L-tryptophan, hydroxylysine, and L-aspartate. The marker may be applied to a prediction model to predict a possibility of a subject with diabetes. The diabetes herein includes type 1 diabetes, type 2 diabetes, or GDM. In some embodiments, the diabetes is GDM. The GDM is defined as a glucose tolerance disorder first diagnosed during pregnancy. Mothers with GDM are at higher risk for gestational hypertension and pre-eclampsia, and fetuses of mothers with GDM may have increased birth weight (e.g., macrosomia), thus increasing the risk of obstructed shoulder birth, which is a serious adverse outcome of labor. In addition, the GDM promotes the development of metabolic complications, including obesity, metabolic syndrome, type 2 diabetes mellitus (T2DM), and cardiovascular disease in mothers and offspring later in life. As a result, the GDM adds a significant burden to pregnant women, fetuses, and society worldwide.

According to the 2014 Chinese GDM guidelines, based on the IADPSG criteria and the International Diabetes Federation (IDF), a “one-step” 2-hour, 75-g oral glucose tolerance test (OGTT) is recommended for all pregnant women at 24 to 28 weeks of gestation. However, the OGTT has some drawbacks, e.g., t an overnight fast of at least 8 hours; the drinking a liquid containing 75 g of glucose within 5 minutes; some pregnant women have difficulty in tolerating glucose drinks, which may cause adverse effects, including nausea, vomiting, bloating and headache. Thus, the OGTT cannot be easily applied to many pregnant women. In addition, a study based on 3098 Chinese pregnant women found that 75.8% of normoglycemic women had to undergo OGTT without any clinical benefit. A two-step test is commonly used in the United States, with a non-fasting 50 g screening test followed by a 100 g OGTT for those whose screen result is positive. Only high-risk women receive a diagnostic 75 g OGTT, which is promoted by the national health system in Italian. In the present disclosure, the risk of a subject with diabetes can be predicted by a prediction model based on a concentration of a marker in a sample from the subject without overnight fasting and without oral glucose, which is physically friendly to the subject and does not cause adverse reactions to the subject, and is more objective and convenient.

As used in the present disclosure, the “subject” (which may also be referred to as “individual”, “person”) is a subject undergoing a diabetes test or prediction. In some embodiments, the subject may be a vertebrate animal. In some embodiments, the vertebrate animal is a mammal. The mammal includes, but is not limited to, a primate (including human and non-human primates) and a rodent (e.g., mice and rats). In some embodiments, the subject may be a human. In some embodiments, the subject is a pregnant woman.

According to an aspect of the present disclosure, there is provided a marker for predicting a possibility of a subject with diabetes. The diabetes may include type I diabetes, type II diabetes, or GDM. In some embodiments, the diabetes may be type I diabetes. In some embodiments, the diabetes may be type II diabetes. In some embodiments, the diabetes may be GDM.

In some embodiments, the marker may be related to diabetes-related metabolism, e.g., metabolism related to insulin resistance, gut microbial metabolism, glycerophospholipid metabolism, etc. In some embodiments, the marker may include a glucose analogue, an organic acid, an organic compound, an amino acid, or the like. In some embodiments, the glucose analogue may include 1,5-AG. The organic acid may include α-HB. The organic compound may include ethanolamine, trimethylamine oxide (TMAO). The amino acid may include L-phenylalanine, L-tryptophan, L-tyrosine, L-isoleucine, L-leucine, L-valine, citrulline, cystine, glutamine, glutamic acid, hydroxylysine, L-aspartic acid, L-alanine, L-proline, L-threonine, lysine, methionine, taurine, or the like. In some embodiments, the marker may also include other compounds, such as ADMA, symmetric dimethylarginine (SDMA), oleic acid (OA), linoleylglycerophosphocholine (LPGC), etc.

In some embodiments, the marker may include at least one of α-HB, 1,5-AG, ADMA, cystine, ethanolamine, taurine, L-leucine, L-tryptophan, hydroxylysine, L-aspartate. In some embodiments, the marker may be α-HB. In some embodiments, the marker may include at least one of 1,5-AG and ADMA. In some embodiments, the marker may include all of 1,5-AG and ADMA. In some embodiments, the marker may include at least one of cystine, ethanolamine, taurine, L-leucine, L-tryptophan, and hydroxylysine. In some embodiments, the marker may include all of cystine, ethanolamine, taurine, L-leucine, L-tryptophan, and hydroxylysine. In some embodiments, the marker may include at least one of α-HB, 1,5-AG, cystine, ethanolamine, taurine, L-aspartic acid. In some embodiments, the marker may include all of α-HB, 1,5-AG, cystine, ethanolamine, taurine, and L-aspartate.

In some embodiments, the marker described above may be applied as a variable of a prediction model. The prediction model may include multiple prediction models, e.g., prediction models 2-5 in embodiments. Each prediction model may be related to at least one of the aforementioned markers (e.g., as a variable of the prediction model). In some embodiments, prediction model 2 may be related to α-HB. In some embodiments, prediction model 3 may be related to 1,5-AG and ADMA. In some embodiments, prediction model 4 may be related to cystine, ethanolamine, taurine, L-leucine, L-tryptophan, and hydroxylysine. In some embodiments, prediction model 5 may be related to α-HB, 1,5-AG, cystine, ethanolamine, taurine, and L-aspartic acid. In some embodiments, the prediction model may also include other variables, e.g., a conventional variable (e.g., an age and BMI of the subject). In some embodiments, the prediction models 2-5 may also be related to the subject's age and BMI. In some embodiments, the prediction model may also include prediction model 1, which is only related to the subject's age and BMI. It should be noted that for subjects who are pregnant women, the BMI is a pre-pregnancy BMI. In some embodiments, the prediction model may also be a model that integrates multiple prediction models as described above.

The prediction model may output a probability value based on the concentrations of the aforementioned markers to predict the possibility of the subject with diabetes. Specifically, these markers may be used as variables of the relevant prediction model, and the concentrations of the markers of the subject is input into the relevant prediction model. The prediction model may output a probability value, and the probability value is compared to a threshold corresponding to a model to determine the possibility of the subject with diabetes. If the probability value is greater than or equal to the threshold, the subject is predicted to be more likely to have diabetes. Otherwise, the subject is predicted to be less likely to have diabetes.

According to another aspect of the present disclosure, there is provided a use of a marker in preparing a reagent, composition or kit for predicting a possibility of a subject with diabetes. The prediction includes the following steps.

Based on a sample from a subject, a concentration of a marker is determined, wherein the marker includes at least one of α-HB, 1,5-AG, ADMA, ethanolamine, taurine, L-leucine, L-tryptophan, hydroxylysine, and L-aspartic acid.

A possibility of the subject with diabetes is predicted by using a prediction model related to the marker based on the concentration of the marker.

In some embodiments, the subject may be an individual with or without diabetes. In some embodiments, the subject may be a pregnant woman. The sample of the subject may be a serum sample, a plasma sample, a saliva sample, a urine sample, etc. In some embodiments, the sample may be a serum sample or a plasma sample.

In some embodiments, the marker described herein includes the marker described above. In some embodiments, the marker may include at least one of α-HB, 1,5-AG, ADMA, cystine, ethanolamine, taurine, L-leucine, L-tryptophan, hydroxylysine, L-aspartic acid.

In some embodiments, the marker may be α-HB. In some embodiments, the marker may include at least one of 1,5-AG and ADMA. The marker may include all of 1,5-AG and ADMA. In some embodiments, the marker may include at least one of cystine, ethanolamine, taurine, L-leucine, L-tryptophan, and hydroxylysine. In some embodiments, the marker may include all of cystine, ethanolamine, taurine, L-leucine, L-tryptophan, and hydroxylysine. In some embodiments, the marker may include at least one of α-HB, 1,5-AG, cystine, ethanolamine, taurine, L-aspartic acid. In some embodiments, the marker may include all of α-HB, 1,5-AG, cystine, ethanolamine, taurine, and L-aspartate.

In some embodiments, the concentration of the marker in the sample may be measured by mass spectrometry (e.g., liquid chromatography-mass spectrometry (LC-MS), gas chromatography-mass spectrometry (GC-MS), matrix-assisted laser desorption/ionization time-of-flight mass spectrometry (MALDI-TOF MS), immunoassay, enzymatic assay, etc. In some embodiments, the concentration of the marker may be determined by LC-MS. The method for determining the concentration of the marker can be referred to the “determination of metabolite concentration” section in the embodiments.

In some embodiments, the variables of the different prediction models may include different markers. Each prediction model may be related to at least one of the aforementioned markers. In some embodiments, the prediction models may include multiple prediction models, e.g., the prediction models 2-5 of the embodiments. Each prediction model may be related to at least one of the above-mentioned markers. In some embodiments, the prediction model 2 may be related to α-HB. In some embodiments, the prediction model 3 may be related to 1,5-AG and ADMA. In some embodiments, the prediction model 4 may be related to cystine, ethanolamine, taurine, L-leucine, L-tryptophan, and hydroxylysine. In some embodiments, the prediction model 5 may be related to α-HB, 1,5-AG, cystine, ethanolamine, taurine, and L-aspartic acid. In some embodiments, the prediction model may also include other variables, e.g., a conventional variable (e.g., an age and BMI of the subject). In some embodiments, the prediction model may further include the prediction model 1, which is related to the age and BMI of the subject. In some embodiments, the prediction model may further include a model that integrates the multiple prediction models described above.

$$log ( \frac{p}{1 - p} ) = - 13.38647 + 1.49950 * ( α - H B ) + 0.0 \; 7665 * age + 0.11713 * BMI$$

In some embodiments, the prediction model (e.g., prediction model 2) may be represented by equation (1):

$$log ( \frac{p}{1 - p} ) = - 13.38647 + 1.49950 * ( α - HB ) + 0.0 \; 7665 * age + 0.11713 * BMI \qquad ( 1 )$$

In some embodiments, the prediction model (e.g., prediction model 3) may be represented by equation (2):

$$log ( \frac{p}{1 - p} ) = - 3.5 \; 6131 + ( - 0.74606 ) * ( 1 , 5 - AG ) + ( - 1.40508 ) * ADMA + 0.07688 * age + 0.12063 * BMI \qquad ( 2 )$$

In some embodiments, the prediction model (e.g., prediction model 4) may be represented by equation (3):

$$log ( \frac{p}{1 - p} ) = - 6.98386 + 1.5 \; 6579 * cystine + ( 5.25949 ) * ethanolamine + 1.64365 * ( L - leucine ) + ( - 1.80619 ) * ( L - tryptophan ) + 0.7315 * hydroxylysine + 2.47105 * taurine + 0.08815 * age + 0.12894 * BMI \qquad ( 3 )$$

In some embodiments, the prediction model (e.g., prediction model 5) may be represented by equation (4):

$$log ( \frac{p}{1 - p} ) = - 6.3 \; 3027 + ( - 0.81716 ) * ( 1 , 5 - AG ) + 1.43266 * ( α - HB ) + 1.51073 * taurine + 0.9601 * ( L - aspartate ) + 1.26682 * cystine + ( - 5.1819 ) * ethanolamine + 0.0787 * age + 0.127 * BMI \qquad ( 4 )$$

In the above equations, the p value is the probability value that the subject is diabetic,

$$log ( \frac{p}{1 - p} )$$

is the odds ratio, and the name of each marker indicates the concentration of each marker in μmol/L. The unit μmol/L here is only exemplary and may also be other concentration units known to those in the art, e.g., mol/L, ug/mL, g/L, etc., and the present disclosure does not limit this. It should be noted that for subjects who are pregnant women, the BMI in the above equations is the pre-pregnancy BMI.

In some embodiments, the prediction model may be obtained by a model training. A training set may be used to train an initial model to obtain a trained model. The training set may include a concentration of a marker of a sample, a regular feature of the subject (e.g., age, BMI), a classification data of whether the sample subject has diabetes (e.g., gestational diabetes). In some embodiments, a validation set may be used to validate the trained model and to continuously adjust parameters of the trained model. In some embodiments, the validation set may be used to validate the prediction model.

In some embodiments, the prediction model may be constructed by logistic regression, support vector machine (SVM), Bayesian classifier, K-nearest neighbor (KNN), decision tree, or any combination thereof. In some embodiments, the prediction model may be a logistic regression model.

A receiver operating characteristic (ROC) curve may be used to evaluate the performance of a prediction model. The ROC curve may indicate a prediction capability of the prediction model. The ROC curve is a curve plotted with a sensitivity (true positive rate) as a vertical coordinate and a specificity (true negative rate) as a horizontal coordinate. Area under the curve (AUC) may be determined based on the ROC curve, and the AUC may be used to indicate the accuracy of the prediction model; the higher the AUC, the higher the accuracy of the prediction model.

In some embodiments, the AUC of the prediction model may be greater than 0.7. In some embodiments, the AUC of the prediction model may be greater than 0.75. In some embodiments, the AUC of the prediction model may be greater than 0.8. In some embodiments, the AUC of the prediction model may be greater than 0.85. In some embodiments, the AUC of the prediction model may be greater than 0.9. Specifically, in some embodiments, the AUC of the prediction model 2 may be greater than 0.7. In some embodiments, the AUC of prediction model 2 may be greater than 0.7. In some embodiments, the AUC of the prediction model 3 may be greater than 0.75. In some embodiments, the AUC of the prediction model 4 may be greater than 0.85. In some embodiments, the AUC of the prediction model 5 may be greater than 0.85. In some embodiments, the AUC of the prediction model 5 may be greater than 0.9. In some embodiments, the prediction models 2-5 all have AUCs greater than 0.7, all with some accuracy, but the prediction models 2-5 may have different AUCs. For example, the AUCs of prediction models 2-5 are in an increasing order, i.e., the accuracy of the prediction model 5 is better than the accuracy of the prediction model 4, the accuracy of the prediction model 4 is better than the accuracy of the prediction model 3, the accuracy of the prediction model 3 is better than the accuracy of the prediction model 2.

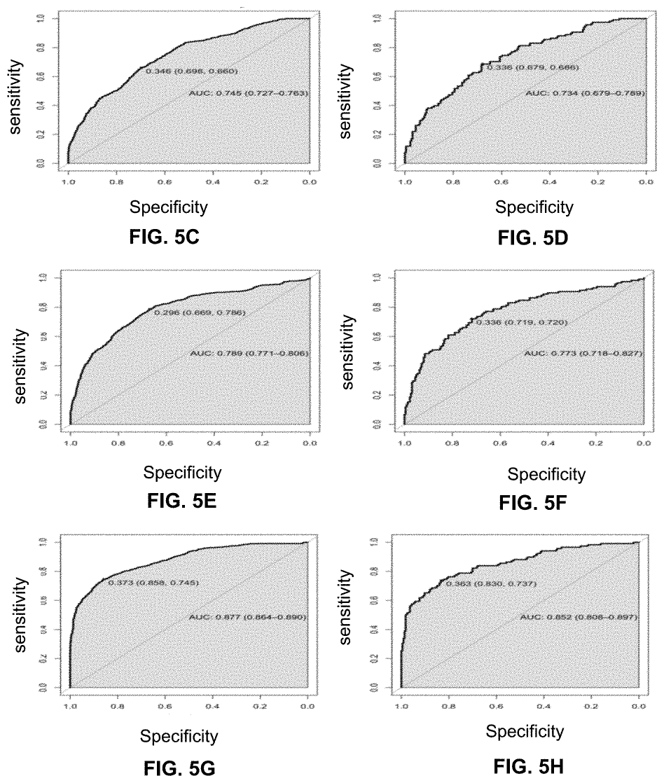

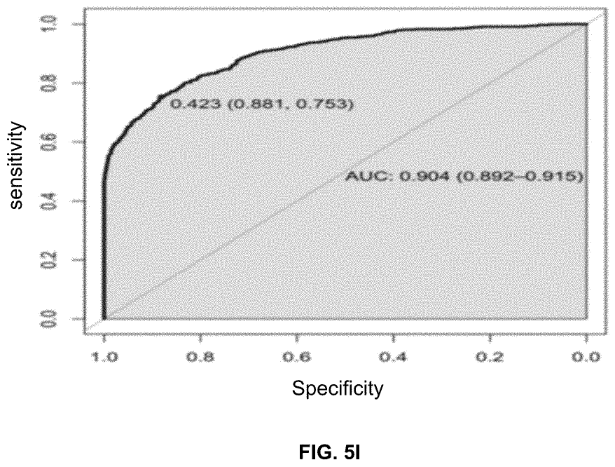

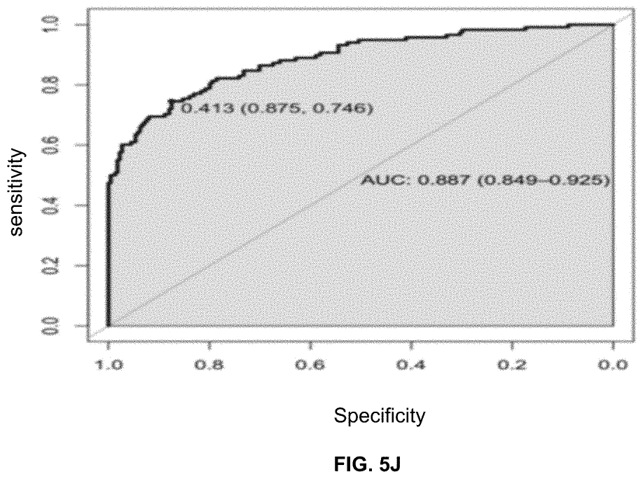

FIGS. 5C-5J illustrate the ROCs of the prediction models 2-5 in the training set and validation set, respectively, according to some embodiments of the present disclosure. Exemplarily, the prediction model 2 has an AUC of 0.734 in the validation set, the prediction model 3 has an AUC of 0.773 in the validation set, the prediction model 4 has an AUC of 0.852 in the validation set, and the prediction model 5 has an AUC of 0.887 in the validation set.

In some embodiments, the sensitivity of the prediction model may be greater than 65%. In some embodiments, the sensitivity of the prediction model may be greater than 70%. In some embodiments, the sensitivity of the prediction model may be greater than 75%. In some embodiments, the sensitivity of the predictive model may be greater than 80%. In some embodiments, the sensitivity of the prediction model may be greater than 85%. In some embodiments, the sensitivity of the prediction model may be greater than 90%. Specifically, in some embodiments, the sensitivity of the prediction model 2 may be greater than 65%. In some embodiments, the sensitivity of the prediction model 2 may be greater than 65%. In some embodiments, the sensitivity of the prediction model 3 may be greater than 70%. In some embodiments, the sensitivity of the prediction model 4 may be greater than 70%. In some embodiments, the sensitivity of the prediction model 5 may be greater than 70%.

In some embodiments, the specificity of the prediction model may be greater than 65%. In some embodiments, the specificity of the prediction model may be greater than 70%. In some embodiments, the specificity of the prediction model may be greater than 75%. In some embodiments, the specificity of the prediction model may be greater than 80%. In some embodiments, the specificity of the prediction model may be greater than 85%. In some embodiments, the specificity of the prediction model may be greater than 90%. Specifically, in some embodiments, the specificity of the prediction model 2 may be greater than 65%. In some embodiments, the specificity of the prediction model 3 may be greater than 70%. In some embodiments, the specificity of the prediction model 4 may be greater than 80%. In some embodiments, the specificity of the prediction model 5 may be greater than 85%.

FIGS. 5C-5J illustrate the ROCs of the prediction models 2-5 in the training and validation sets, respectively, according to some embodiments of the present disclosure. Exemplarily, the sensitivity of the prediction model 2 is 68.6% and the specificity of the prediction model 2 is 67.9% in the validation set; the sensitivity of the prediction model 3 is 72% and the specificity of the prediction model 3 is 71.9% in the validation set; the sensitivity of the prediction model 4 is 73.7% and the specificity of the prediction model 4 is 83%; the sensitivity of the prediction model 5 is 74.6% and the specificity of the prediction model 5 is 87.5% in the validation set.

For more information about the prediction model, please refer to the “determination of the prediction model” of Examples.

In some embodiments, the predicting the possibility of the subject with diabetes using a prediction model related to at least one of the markers based on the concentration of at least one of the markers may include: inputting the concentration of the marker corresponding to each prediction model and outputting a prediction value. By comparing the prediction value with a threshold, the possibility of the subject with diabetes may be predicted for the subject. In the case of the prediction model 5, for example, the concentration (in μmol/L) of the marker related to the prediction model 5 is input to equation (4), the prediction model 5 may output a prediction value (i.e., probability value p), and compare it with a threshold corresponding to the prediction model 5, thereby predicting the possibility of the subject with diabetes.

In some embodiments, the threshold of the prediction model may be a threshold calculated by a Youden's index. For example, considering only the 2 indexes, sensitivity and specificity, the threshold on the ROC curve may be calculated using the Youden's index. In some embodiments, the threshold of the prediction model 2 is 0.336. In some embodiments, the threshold of the prediction model 3 is 0.336. In some embodiments, the threshold of the prediction model 4 is 0.363. In some embodiments, the threshold of the prediction model 5 is 0.413.

In some embodiments, the threshold of the prediction model may be any value in a selected threshold range. In some embodiments, the threshold range may be determined based on a range of sensitivities and specificities. For example, the threshold range is selected based on a range of sensitivities and specificities. The threshold value of the prediction model may be determined from the threshold range. In some embodiments, the threshold range corresponding to a sensitivity and specificity of the prediction model 5 at [0.8, 0.85] may be selected, for example, [0.288597,0.323644]. In some embodiments, the threshold range corresponding to the sensitivity and specificity of the prediction model 4 at [0.75, 0.8] may be selected, e.g., [0.274613,0.323241]. In some embodiments, the threshold range corresponding to the sensitivity and specificity of the prediction model 3 at [0.7, 0.75] may be selected, e.g., [0.317268,0.360159]. In some embodiments, the threshold range corresponding to the sensitivity and specificity of the prediction model 2 at [0.65, 0.7] may be selected, e.g., [0.309508,0.374544].

In some embodiments, if the prediction value described is greater than or equal to the threshold described, the possibility of the subject with diabetes may be relatively high. If the prediction value is less than the threshold, the possibility of the subject with diabetes may be relatively low. A relatively high possibility of a subject with diabetes means that a probability of a subject with diabetes is greater than or equal to 80%, 85%, 90%, 95%, 98%, or 100%. In some embodiments, a relatively high possibility of a subject with diabetes refers to a subject with diabetes. A relatively low possibility of a subject with diabetes means that a probability of a subject not with diabetes is greater than or equal to 80%, 85%, 90%, 95%, 98%, or 100%. In some embodiments, a relatively low possibility of a subject with diabetes refers to the subject not with diabetes.

For more information about the prediction model predicting a possibility of a subject with diabetes, please refer to the “Application of the prediction model” of the Examples.

According to a further aspect of the present disclosure, there is provided a use of a prediction model in preparing a reagent, composition or kit for predicting a possibility of a subject with diabetes. The prediction model may be related to the marker. In some embodiments, the marker may include at least one of α-HB, 1,5-AG, ADMA, cystine, ethanolamine, taurine, L-leucine, L-tryptophan, hydroxylysine, L-aspartate. In some embodiments, the prediction model may include multiple prediction models, e.g., prediction models 2-5 in Examples. Each prediction model may be related to at least one of the above-mentioned markers (e.g., as a variable of the prediction model). In some embodiments, the prediction model 2 may be related to α-HB. In some embodiments, the prediction model 3 may be related to 1,5-AG and ADMA. In some embodiments, the prediction model 4 may be related to cystine, ethanolamine, taurine, L-leucine, L-tryptophan, and hydroxylysine. In some embodiments, the prediction model 5 may be related to α-HB, 1,5-AG, cystine, ethanolamine, taurine, and L-aspartic acid. In some embodiments, the prediction model may also include other variables, e.g., a conventional variable (e.g., an age and BMI of the subject). In some embodiments, the prediction model may further include prediction model 1, which is related to the age and BMI of the subject. In some embodiments, the prediction model may further include a model that integrates multiple prediction models as described above. In some embodiments, the prediction models 2-5 are represented by equations (1)-(4), respectively, as described above. It should be noted that for subjects who are pregnant women, the BMI is the pre-pregnancy BMI.

In some embodiments, the prediction model may be constructed by logistic regression, support vector machine (SVM), Bayesian classifier, K-nearest neighbor (KNN), a decision tree, or the like, or any combination thereof. In some embodiments, the prediction model may be a logistic regression model.

In some embodiments, the AUC of the prediction model may be greater than 0.7. In some embodiments, the AUC of the prediction model may be greater than 0.75. In some embodiments, the AUC of the prediction model may be greater than 0.8. In some embodiments, the AUC of the prediction model may be greater than 0.85. In some embodiments, the AUC of the prediction model may be greater than 0.9. Specifically, in some embodiments, the AUC of the prediction model 2 may be greater than 0.7. In some embodiments, the AUC of prediction model 2 may be greater than 0.7. In some embodiments, the AUC of the prediction model 3 may be greater than 0.75. In some embodiments, the AUC of the prediction model 4 may be greater than 0.85. In some embodiments, the AUC of the prediction model 5 may be greater than 0.85. In some embodiments, the AUC of the prediction model 5 may be greater than 0.9. In some embodiments, the prediction models 2-5 all have AUCs greater than 0.7, all with some accuracy, but the prediction models 2-5 may have different AUC values. For example, the AUCs of the prediction models 2-5 are in an increasing order, i.e., the accuracy of prediction model 5 is better than the accuracy of prediction model 4, the accuracy of prediction model 4 is better than the accuracy of prediction model 3, the accuracy of prediction model 3 is better than the accuracy of prediction model 2.

FIGS. 5C-5J illustrate the ROCs of the prediction models 2-5 in the training and validation sets, respectively, according to some embodiments of the present disclosure. Exemplarily, the AUC of the prediction model 2 is 0.734 in the validation set, the AUC of the prediction model 3 is 0.773 in the validation set, the AUC of the prediction model 4 is 0.852 in the validation set, and the AUC of the prediction model 5 is 0.887 in the validation set.

In some embodiments, the sensitivity of the predictive model may be greater than 65%. In some embodiments, the sensitivity of the prediction model may be greater than 70%. In some embodiments, the sensitivity of the prediction model may be greater than 75%. In some embodiments, the sensitivity of the predictive model may be greater than 80%. In some embodiments, the sensitivity of the prediction model may be greater than 85%. In some embodiments, the sensitivity of the prediction model may be greater than 90%. Specifically, in some embodiments, the sensitivity of the prediction model 2 may be greater than 65%. In some embodiments, the sensitivity of the prediction model 2 may be greater than 65%. In some embodiments, the sensitivity of the prediction model 3 may be greater than 70%. In some embodiments, the sensitivity of the prediction model 4 may be greater than 70%. In some embodiments, the sensitivity of the prediction model 5 may be greater than 70%.

In some embodiments, the specificity of the prediction model may be greater than 65%. In some embodiments, the specificity of the prediction model may be greater than 70%. In some embodiments, the specificity of the prediction model may be greater than 75%. In some embodiments, the specificity of the prediction model may be greater than 80%. In some embodiments, the specificity of the prediction model may be greater than 85%. In some embodiments, the specificity of the prediction model may be greater than 90%. Specifically, in some embodiments, the specificity of the prediction model 2 may be greater than 65%. In some embodiments, the specificity of the prediction model 3 may be greater than 70%. In some embodiments, the specificity of the prediction model 4 may be greater than 80%. In some embodiments, the specificity of the prediction model 5 may be greater than 85%.

FIGS. 5C-5J illustrate the ROCs of the prediction models 2-5 in the training and validation sets, respectively, according to some embodiments of the present disclosure. Exemplarily, the sensitivity of the prediction model 2 is 68.6% and the specificity of the prediction model 2 is 67.9% in the validation set; the sensitivity of the prediction model 3 is 72% and the specificity of the prediction model 3 is 71.9% in the validation set; the sensitivity of the prediction model 4 is 73.7% and the specificity of the prediction model 4 is 83%; the sensitivity of the prediction model 5 is 74.6% and the specificity of the prediction model 5 is 87.5% in the validation set.

The prediction models constructed in the present disclosure all have good accuracy in accurately predicting whether a subject is diabetic. For more information about the prediction models, please refer to elsewhere described in the present disclosure and is not repeated herein.

According to a further aspect of the present disclosure, there is provided a method for treating diabetes.

Based on a sample from a subject, a concentration of a marker is determined, wherein the marker includes at least one of α-hydroxybutyric acid, 1,5-anhydroglucitol, asymmetric dimethylarginine, cystine, ethanolamine, taurine, L-leucine, L-tryptophan, hydroxylysine, and L-aspartic acid. In some embodiments, the marker may include at least one of α-HB, 1,5-AG, ADMA, cystine, ethanolamine, taurine, L-leucine, L-tryptophan, hydroxylysine, L-aspartate. In some embodiments, the marker may be α-HB. In some embodiments, the marker may include at least one of 1,5-AG and ADMA. The marker may include all of 1,5-AG and ADMA. In some embodiments, the marker may include at least one of cystine, ethanolamine, taurine, L-leucine, L-tryptophan, and hydroxylysine. In some embodiments, the marker may include all of cystine, ethanolamine, taurine, L-leucine, L-tryptophan, and hydroxylysine. In some embodiments, the marker may include at least one of α-HB, 1,5-AG, cystine, ethanolamine, taurine, L-aspartic acid. In some embodiments, the marker may include all of α-HB, 1,5-AG, cystine, ethanolamine, taurine, and L-aspartate.

In some embodiments, the concentration of the marker in the sample may be determined by mass spectrometry (e.g., liquid chromatography-mass spectrometry, gas chromatography-mass spectrometry, matrix-assisted laser desorption/ionization time-of-flight mass spectrometry), immunoassay, enzymatic assay, or the like. In some embodiments, the concentration of the marker may be determined by liquid chromatography-mass spectrometry.

A possibility of the subject with diabetes is predicted by using a prediction model related to the marker based on the concentration of the marker.

In some embodiments, the prediction models described above (e.g., prediction models 2-5) may be used to predict the possibility of the subject with diabetes. For more information about this step, please refer to the description above and is not repeated herein.

If a prediction result is that the subject has diabetes (e.g., the prediction model outputs a probability value greater than or equal to a corresponding threshold), different treatments may be taken for different subjects.

In some embodiments, if the subject is a pregnant woman and the prediction result is that the subject has diabetes, the subject is further diagnosed using an OGTT, and if the OGTT result also indicates that the subject has diabetes, the subject may be administered a drug to treat the diabetes. The prediction model of the present disclosure can screen out non-GDM pregnant women who do not need to do OGTT, thereby reducing the pain and inconvenience of OGTT for pregnant women. The prediction result of the prediction model can provide a reliable and accurate reference for subsequent diagnosis and treatment.

In some embodiments, if the subject is a non-pregnant woman, and the prediction result is that the subject has diabetes, a drug may be administered to the subject to treat the diabetes. In some embodiments, if the subject is a pregnant woman, a follow-up diagnosis (e.g., OGTT) may be performed on the subject to further confirm the diagnosis before administering the drug for diabetes to the subject.

In some embodiments, the drug for treating diabetes may include insulin, sulfonylurea agonists, nonsulfonylurea agonists, biguanides, alpha-glucosidase inhibitors (e.g., acarbose (Glucobay®)), thiazolidinediones (e.g., pioglitazone, rosiglitazone maleate), or the like. The sulfonylurea agonists may include glibenclamide, glipizide, gliclazide, glipizide, glimepiride, etc. The nonsulfonylurea agonists may include repaglinide (NovoNorm®), nateglinide (Glinate®), etc. The biguanides may include metformin extended-release tablets, metformin etc.

According to a further aspect of the present disclosure, there is provided a system for predicting a possibility of a subject with diabetes. The system may include: an acquisition module, a training module, and a prediction module.

The acquisition module may be used to obtain a concentration of a marker of a subject sample. The marker may include at least one of α-HB, 1,5-AG, ADMA, cystine, ethanolamine, taurine, L-leucine, L-tryptophan, hydroxylysine, L-aspartate. In some embodiments, the marker may be alpha-HB. In some embodiments, the marker may include at least one of 1,5-AG and ADMA. The marker may include all of 1,5-AG and ADMA. In some embodiments, the marker may include at least one of cystine, ethanolamine, taurine, L-leucine, L-tryptophan, and hydroxylysine. In some embodiments, the marker may include all of cystine, ethanolamine, taurine, L-leucine, L-tryptophan, and hydroxylysine. In some embodiments, the marker may include at least one of α-HB, 1,5-AG, cystine, ethanolamine, taurine, L-aspartic acid. In some embodiments, the marker may include all of α-HB, 1,5-AG, cystine, ethanolamine, taurine, L-aspartate. The acquisition module may also be used to obtain a conventional feature of the subject, e.g., an age, a BMI, a height, a weight, etc.

The training module may be used to train an initial model using a training set to obtain a prediction model. In some embodiments, the training module may be used to train the initial model using the training set to obtain multiple prediction models, e.g., prediction models 2-5. The prediction model is related to at least one of the markers, e.g., the prediction models 2-5 are related to different markers, as described. The prediction model may also be related to the age and BMI of the subject. In some embodiments, the prediction model 2 may be related to α-HB. In some embodiments, the prediction model 3 may be related to 1,5-AG and ADMA. In some embodiments, the prediction model 4 may be related to cystine, ethanolamine, taurine, L-leucine, L-tryptophan, and hydroxylysine. In some embodiments, the prediction model 5 may be related to α-HB, 1,5-AG, cystine, ethanolamine, taurine, and L-aspartic acid. For more information about the prediction model, please refer to the description elsewhere in the present disclosure and is not repeated herein.

The prediction module may be used to predict a possibility of the subject with diabetes using a prediction model based on a concentration of at least one of the markers. For example, the concentration of the marker corresponding to the prediction model is input into the prediction model, and the prediction model may output a prediction value. Comparing the prediction value with a threshold of the prediction model, the prediction module may predict that the possibility of the subject with diabetes is high when the prediction value is greater than or equal to the threshold; and the prediction module may predict that the possibility of the subject with diabetes is low when the prediction value is less than the threshold.

It should be understood that the system and its modules for predicting a possibility of a subject with diabetes may be implemented using various means. For example, in some embodiments, the system and its modules may be implemented by hardware, software, or a combination of software and hardware. The hardware may be implemented using a specialized logic; the software may be stored in memory and executed by an appropriate instruction execution system, such as a microprocessor or specially designed hardware. Those skilled in the art can understand that the methods and systems described above may be implemented using computer-executable instructions and/or contained in processor control codes, such as those provided on carrier media such as disks, CDs or DVD-ROMs, programmable memories such as read-only memories (firmware), or data carriers such as optical or electronic signal carriers. The system of the present disclosure and its modules may be implemented not only with hardware circuitry such as ultra-large-scale integrated circuits or gate arrays, semiconductors such as logic chips, transistors, etc., or programmable hardware devices such as field-programmable gate arrays, programmable logic devices, etc., but also with software executed, for example, by various types of processors, and also by a combination of the above hardware and software (e.g., firmware) to implement.

### EXAMPLES

### Significance Tests for Clinical Variables of GDM and Non-GDM Groups

In this study, 369 subjects (e.g., pregnant women) were subjected to an OGTT with 75 g of anhydrous glucose in solution. These subjects were divided into two groups, the GDM group and the non-GDM group, based on the test results. The subjects in both groups were also tested for the clinical variables shown in Table 1 below, and statistical tests of significance were performed to identify variables that were significantly different in the two groups. The statistical test of significance used in age, systolic and diastolic blood pressure was the Student's t-test, and the statistical test of significance used in other clinical variables was the Mann-Whitney U test. p value less than 0.05 was considered as significant.

TABLE 1
Clinical features of the GDM and non-GDM groups

| | Number of | | Non-GDM | | GDM | | |
|---|---|---|---|---|---|---|---|
| Variables | people (n = 369) | | (n = 241) | | (n = 128) | | P |
| Age (years) | 31.14 | (4.78) | 30.31 | (4.41) | 32.74 | (5.06) | <0.001 |
| Pre-pregnancy weight (kg) | 55.00 | (50.00-61.50) | 54.00 | (50.00-60.00) | 59.00 | (53.00-66.75) | <0.001 |
| Pre-pregnancy BMI (kg/m₂) | 21.46 | (19.57-23.71) | 20.76 | (19.09-22.78) | 22.72 | (20.61-25.87) | <0.001 |
| Systolic pressure (mm Hg) | 115.41 | (12.51) | 114.02 | (11.48) | 118.13 | (13.97) | 0.006 |
| Diastolic pressure (mm Hg) | 70.87 | (9.57) | 69.75 | (9.61) | 73.06 | (9.12) | 0.002 |
| Triglyceride (mmol/L) | 1.44 | (1.11-1.98) | 1.39 | (1.08-1.91) | 1.55 | (1.23-2.05) | 0.012 |
| Total cholesterol (mmol/L) | 4.90 | (4.40-5.49) | 4.85 | (4.37-5.47) | 4.95 | (4.46-5.51) | 0.713 |
| High-density lipoprotein | 1.74 | (1.50-1.96) | 1.79 | (1.53-1.99) | 1.63 | (1.44-1.89) | 0.006 |
| cholesterol (mmol/L) | | | | | | | |
| Low-density lipoprotein | 2.49 | (2.12-2.98) | 2.49 | (2.13-3.00) | 2.50 | (2.12-2.98) | 0.727 |
| cholesterol (mmol/L) | | | | | | | |
| Fasting glucose (mmol/L) | 4.46 | (4.20-4.85) | 4.33 | (4.13-4.56) | 4.97 | (4.54-5.35) | <0.001 |
| 1 h glucose (mmol/L) | 8.03 | (6.50-9.30) | 7.41 | (6.16-8.37) | 10.06 | (8.82-10.62) | <0.001 |
| 2 h glucose (mmol/L) | 6.70 | (5.73-7.97) | 6.21 | (5.41-7.07) | 8.61 | (7.10-9.36) | <0.001 |
| 3 h glucose (mmol/L) | 5.66 | (4.63-6.60) | 5.52 | (4.50-6.26) | 7.08 | (5.78-7.86) | <0.001 |
| Glycated hemoglobin (%) | 5.20 | (5.00-5.40) | 5.20 | (4.90-5.35) | 5.30 | (5.10-5.50) | <0.001 |
| Fasting insulin (pmol/L) | 9.21 | (6.52-12.47) | 9.00 | (6.47-11.70) | 10.98 | (6.68-15.05) | 0.062 |
| 2 h insulin (pmol/L) | 67.38 | (44.39-98.77) | 63.01 | (41.30-91.70) | 94.18 | (59.43-139.00) | <0.001 |
| Indicators of insulin | 1.80 | (1.25-2.53) | 1.72 | (1.24-2.39) | 2.31 | (1.48-3.40) | 0.006 |
| resistance * | | | | | | | |
| Islet cell function | 209.44 | (147.21-299.30) | 222.02 | (161.33-305.68) | 159.63 | (109.57-257.16) | <0.001 |
| indicators (%)* | | | | | | | |

where the above data are the mean (standard deviation) or median (interquartile range); P values are the differences between patients diagnosed with and without GDM; and * indicates log-transformation before analysis.

The results in Table 1 above show that compared to the non-GDM group, the subjects in the GDM group had significantly greater age, pre-pregnancy BMI (p<0.001), significantly higher blood pressure, triglycerides, glycosylated hemoglobin, and indicators of insulin resistance (p<0.02), and significantly lower high-density lipoprotein cholesterol and islet cell function indicators (both p<0.01), while total cholesterol, low-density lipoprotein cholesterol and fasting insulin were not significantly different (p>0.05).

### Determination of Metabolite Concentration

Metabolite concentrations related to the variables identified above as significantly different (other clinical variables except age and pre-pregnancy BMI) were measured by LC-MS for significant difference analysis.

Specifically, plasma samples were obtained from 369 subjects, and subjected to protein precipitation, shake and centrifugation to obtain the supernatant. The metabolites to be measured were first separated from the supernatant by using ultra performance liquid chromatography. Then, the mass spectrometry isotope internal standard quantification method was used to establish a calibration curve using a concentration ratio of standard sample of the metabolites to internal standard as the X-axis and a peak area ratio of standard sample of the metabolites to internal standard as the Y-axis and thus the content of the relevant metabolites was calculated. However, the conditions of high performance liquid chromatography and mass spectrometry are different for different metabolites, as described below.

I. Detection of 25 Amino Acids and their Derivatives

(1) High Performance Liquid Chromatography Conditions:

Mobile phase A: water (containing 0.1% formic acid);

Mobile phase B: acetonitrile (containing 0.1% formic acid);

Chromatographic column: ACQUITY UPLC BEH C18 (2.1×100 mm, 1.7 μm);

by means of a gradient elution, see Table 2;

flow rate: 0.4 mL/min, a column temperature: 50° C., and an injection volume: 1 μL;

TABLE 2
Mobile phase gradient elution parameters

| | Flow | | | |
|---|---|---|---|---|
| Time(min) | rate(mL/min) | % A | % B | Curve |
| 0.0 | 0.4 | 99 | 1 | — |
| 2.0 | 0.4 | 90 | 10 | 6 |
| 5.0 | 0.4 | 85 | 15 | 6 |
| 7.0 | 0.4 | 2 | 98 | 6 |
| 10.0 | 0.4 | 99 | 1 | 1 |

(2) Mass Spectrometry Conditions:

In the positive ion mode of electrospray ionization, a mass spectrometry scan mode with multiple reaction monitoring was used; the spray voltage was 3.0 kV; the desolvation temperature was 120° C.; the nebulizer gas temperature was 400° C., the nebulizer gas flow rate was 800 L/h and the cone pore gas flow rate was 150 L/h; the metabolites to be measured and their internal standards were monitored simultaneously; the declustering voltage and collision voltage parameters of each metabolite to be measured are shown in Table 3.

TABLE 3
Mass spectral parameters of amino acids and their derivatives

| Amino acids | | MRM | | |
|---|---|---|---|---|
| and their | Internal | monitoring ion | Declustering | Collision |
| derivatives | standards | pairs (Q1/Q3) | voltage (V) | voltage (V) |
| Ethanolamine | Lysine -d4 | 232.1/171.1 | 30.0 | 20.0 |
| Lysine | Lysine -d4 | 244.1/171.1 | 30.0 | 6.0 |
| Glycine | Glycine -d3 | 246.1/171.1 | 30.0 | 20.0 |
| Hydroxylysine | Lysine -d4 | 252.1/171.1 | 30.0 | 12.0 |
| β-Alanine | α-Alanine -d4 | 260.1/171.1 | 30.0 | 20.0 |
| α-Alanine | α-Alanine -d4 | 260.1/171.1 | 30.0 | 20.0 |
| Sarcosine | Lysine -d4 | 264.1/171.1 | 30.0 | 20.0 |
| γ-Aminobutyric | Lysine -d4 | 274.1/171.1 | 30.0 | 20.0 |
| acid | | | | |
| Serine | Valine -d5 | 276.1/171.1 | 30.0 | 20.0 |
| Proline | Valine -d5 | 286.1/171.1 | 30.0 | 6.0 |
| Valine | Valine -d5 | 288.1/171.1 | 30.0 | 8.0 |
| Threonine | Threonine -d5 | 290.1/171.1 | 30.0 | 8.0 |
| Cystine | Cystine -d5 | 290.1/171.1 | 30.0 | 20.0 |
| Taurine | Threonine -d5 | 296.1/171.1 | 30.0 | 20.0 |
| Isoleucine | Isoleucine -d7 | 302.1/171.1 | 30.0 | 6.0 |
| Leucine | Leucine -d7 | 302.1/171.1 | 30.0 | 6.0 |
| Aspartic Acid | Aspartic Acid -d5 | 304.1/171.1 | 30.0 | 20.0 |
| Glutamine | Glutamic acid -d6 | 317.1/171.1 | 30.0 | 8.0 |
| Glutamic acid | Glutamic acid -d6 | 318.1/171.1 | 30.0 | 15.0 |
| Methionine | Methionine -d6 | 320.1/171.1 | 30.0 | 15.0 |
| α-Aminoadipic | Lysine -d4 | 332.1/171.1 | 32.0 | 22.0 |
| acid | | | | |
| Phenylalanine | Phenylalanine -d10 | 336.1/171.1 | 30.0 | 8.0 |
| Arginine | Arginine -d10 | 345.1/171.1 | 40.0 | 30.0 |
| Citrulline | Lysine -d4 | 346.1/171.1 | 30.0 | 12.0 |
| L-Tyrosine | Tyrosine -d10 | 352.1/171.1 | 30.0 | 20.0 |
| L-Tryptophan | Lysine -d4 | 375.1/171.1 | 30.0 | 15.0 |

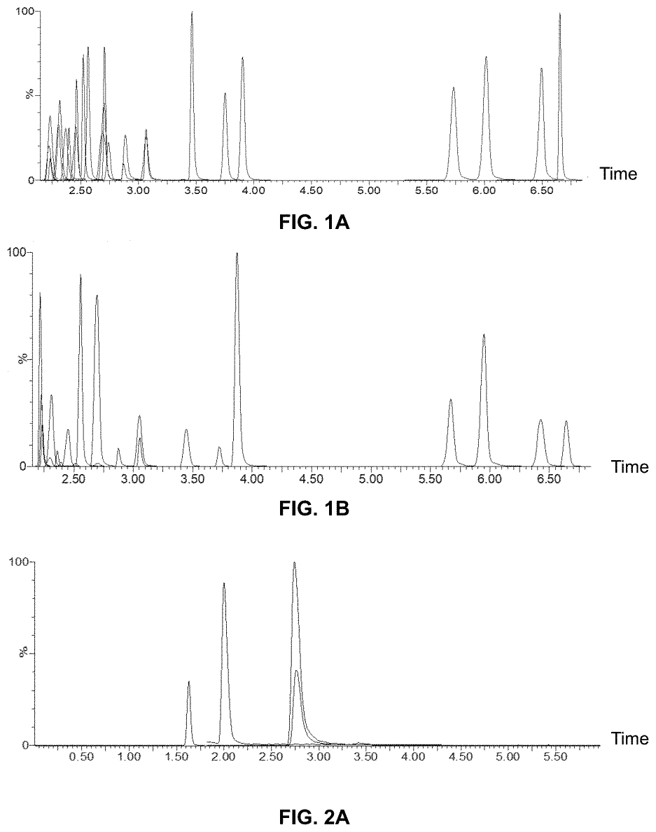

FIG. 1A and FIG. 1B show the total ion chromatograms of 25 amino acids and their derivatives in the standards and plasma samples, respectively. As shown in the figures, the peak shapes of 25 amino acids of the standards and plasma samples and their derivatives were relatively symmetrical and without spurious peak interference, indicating that good detection could be obtained under these conditions.

The isotope internal standard quantification method was used to establish a calibration curve using TargetLynx™ software with a concentration ratio of a standard to an internal standard as the X-axis and a peak area ratio of the standard to the internal standard as the Y-axis. 25 amino acids and their derivatives had good linearity of the linear equations in their respective concentration ranges with correlation coefficients above 0.99, which met the quantitative requirements, as shown in Table 4. Based on the linear equation of the standard curve, the concentrations of the metabolites to be measured in plasma samples were calculated.

TABLE 4
Linear regression equations and linear correlation coefficients
of 25 amino acids and their derivatives

| Amino acids | | | |
|---|---|---|---|
| and their | Curve | | linear |
| derivatives | concentrations (μM) | Linear equations | coefficients(r) |
| Ethanolamine | 0.5-100 | Y = 0.39745X + 0.0394415 | 0.997708 |
| Lysine | 2-400 | Y = 0.114376X + 0.00359747 | 0.999793 |
| Glycine | 2.5-500 | Y = 0.705304X + 4.08366 | 0.99858 |
| Hydroxylysine | 0.05-10 | Y = 0.41125X − 0.00276719 | 0.99883 |
| β-Alanine | 5-1000 | Y = 0.0571702X − 0.001626 | 0.998165 |
| α-Alanine | 5-1000 | Y = 0.0116339X + 0.00147821 | 0.999422 |
| Sarcosine | 0.05-10 | Y = 0.426381X + 0.00759943 | 0.994482 |
| γ-Aminobutyric | 0.05-10 | Y = 0.451118X + 0.0409594 | 0.996159 |
| acid | | | |
| Serine | 2-400 | Y = 0.242328X + 0.0621699 | 0.998019 |
| Proline | 4-800 | Y = 0.0197202X + 0.00923427 | 0.996944 |
| Valine | 2-400 | Y = 0.0373152X − 0.00544172 | 0.999618 |
| Threonine | 2-400 | Y = 0.0812409X − 0.0163731 | 0.999767 |
| Cystine | 0.25-50 | Y = 1.3343X + 0.0320943 | 0.998654 |
| Taurine | 1-200 | Y = 0.139834X + 0.00957988 | 0.999322 |
| Isoleucine | 2-400 | Y = 0.00611762X − 0.00151861 | 0.999111 |
| Leucine | 2-400 | Y = 0.0055017X + 0.00174706 | 0.999103 |
| Aspartic Acid | 0.5-100 | Y = 0.294947X + 0.130759 | 0.997697 |
| Glutamine | 10-2000 | Y = 0.0103302X + 0.0257797 | 0.994306 |
| Glutamic acid | 2-400 | Y = 0.584919X + 0.0174678 | 0.998388 |
| Methionine | 0.5-100 | Y = 0.646694X − 0.0263185 | 0.999871 |
| α-Aminoadipic | 0.05-10 | Y = 0.0901299X − 0.000423814 | 0.993293 |
| acid | | | |
| Phenylalanine | 1-200 | Y = 0.0250056X − 0.00187617 | 0.999788 |
| Arginine | 2-400 | Y = 0.0829017X − 0.0184777 | 0.997229 |
| Citrulline | 1-200 | Y = 0.0367225X + 0.00122589 | 0.998073 |
| Tyrosine | 1-200 | Y = 0.702999X − 0.0633621 | 0.999716 |
| Tryptophan | 0.5-100 | Y = 0.988077X − 0.00636763 | 0.998082 |

II. 1,5-AG, TMAO, ADMA and SDMA Tests

(1) High Performance Liquid Chromatography Conditions:

Mobile phase A: water (containing 0.1% formic acid);

Mobile phase B: acetonitrile (containing 0.1% formic acid);

Chromatographic column: ACQUITY UPLC BEH Amide (2.1×100 mm, 1.7 μm);

by means of a gradient elution, see Table 5;

A flow rate: 0.4 mL/min, a column temperature: 50° C., and an injection volume: 1 μL;

TABLE 5
Mobile phase gradient elution parameters

| | Flow | | | |
|---|---|---|---|---|
| Time(min) | rate(mL/min) | % A | % B | Curve |
| 0.0 | 0.4 | 30 | 70 | — |
| 3.0 | 0.4 | 60 | 40 | 6 |
| 3.5 | 0.4 | 60 | 40 | 6 |
| 6.0 | 0.4 | 30 | 70 | 1 |

(2) Mass Spectrometry Conditions:

A mass spectrometry scan mode with electrospray ionization positive and negative ion switching for multiple reaction monitoring was used; the spray voltage was ESI(+) 3.0 kV/ESI(−) 2.5 kV; the desolvation temperature was 120° C.; the atomization gas temperature was 400° C., the atomization gas flow rate was 800 L/h and the cone pore gas flow rate was 150 L/h; the metabolites to be measured and their internal standards were monitored simultaneously; the declustering voltage and collision voltage of each metabolite to be measured are shown in Table 6.

TABLE 6
Mass spectrometry parameters of metabolites to be measured

| Metabolites | | MRM | | | |
|---|---|---|---|---|---|
| to be | Internal | monitoring ion | Declustering | Collision | |
| measured | standards | pairs (Q1/Q3) | voltage (V) | voltage (V) | ESI(+/−) |
| 1,5-AG | 1,5-AG-¹³C6 | 162.90/100.88 | 10 | 13 | ESI(−) |
| TMAO | TMAO-d9 | 76.2/59.0 | 36 | 11 | ESI(+) |
| ADMA | ADMA-d7 | 203.1→46.0 | 12 | 15 | ESI(+) |
| SDMA | ADMA-d7 | 203.1→172.0 | 12 | 13 | ESI(+) |

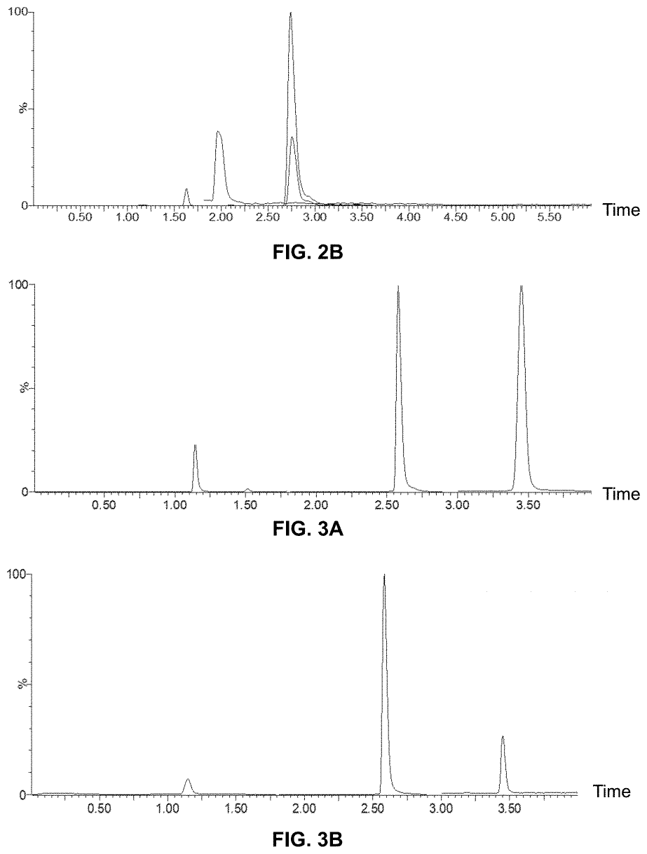

FIG. 2A and FIG. 2B show the total ion chromatograms of standards of 1,5-AG, TMAO, ADMA, and SDMA and the total ion chromatograms of 1,5-AG, TMAO, ADMA, and SDMA in plasma samples, respectively. As shown in the figures, the peak shapes of the standards and plasma samples of 1,5-AG, TMAO, ADMA and SDMA were relatively symmetrical and without spurious peak interference, indicating that good detection could be obtained under these conditions.

The isotope internal standard quantification method was used to establish a calibration curve using TargetLynx™ software with a concentration ratio of metabolite standard to internal standard as the X-axis and a peak area ratio of metabolite standard to internal standard as the Y-axis. 1,5-AG, TMAO, ADMA and SDMA were linearly fitted to the equations in their respective concentration ranges with good linearity and correlation coefficients above 0.99, which met the quantification requirements, see Table 7. Based on the linear method of the standard curve, the concentrations of the substances to be measured in plasma samples were calculated.

TABLE 7
Linear regression equations and linear correlation
coefficients of 1,5-AG, TMAO, ADMA and SDMA

| | Curve | | Linear |
|---|---|---|---|
| Analytes | concentrations(μM) | Linear equations | coefficients (r) |
| 1,5-AG | 4-500 | Y = 0.0299x + 0.0288 | 0.9989 |
| TMAO | 0.4-50 | Y = 0.188x + 0.0339 | 0.9996 |
| ADMA | 0.08-5 | Y = 0.892x + 0.0592 | 0.9990 |
| SDMA | 0.08-5 | Y = 1.11x + 0.0572 | 0.9974 |

III. α-HB, OA and LGPC Tests

(1) High Performance Liquid Chromatography Conditions:

Mobile phase A: water (containing 0.1% formic acid);

Mobile phase B: acetonitrile (containing 0.1% formic acid);

Chromatographic column: ACQUITY UPLC BEH C18 (2.1×50 mm, 1.7 μm);

by means of a gradient elution, see Table 8;

at a flow rate of 0.5 mL/min, a column temperature of 50° C., and an injection volume of 1 μL;

TABLE 8
Mobile phase gradient elution parameters

| Time | Flow rate (mL/min) | % A | % B | Curve |
|---|---|---|---|---|
| 0.0 | 0.5 | 30 | 1 | — |
| 1.0 | 0.5 | 60 | 98 | 6 |
| 3.0 | 0.5 | 60 | 98 | 6 |
| 5.0 | 0.5 | 30 | 1 | 1 |

(2) Mass Spectrometry Conditions:

The mass spectrometry scan mode with electrospray ionization positive and negative ion switching for multiple reaction monitoring was used; the spray voltage was ESI(+) 3.0 kV/ESI(−) 2.5 kV; the desolvation temperature was 120° C.; the atomization gas temperature was 400° C., the atomization gas flow rate was 800 L/h, and the cone pore gas flow rate was 150 L/h; the targets and their internal standards were monitored simultaneously; the declustering voltage and collision voltage parameters of each target are shown in Table 9.

TABLE 9
Target substance spectrum parameters

| | | MRM | | | |
|---|---|---|---|---|---|
| | Internal | monitoring ion | Declustering | Collision | |
| Target | standards | pairs (Q1/Q3) | voltage (V) | voltage (V) | ESI(+/−) |
| α-HB | α-HB-d3 | 102.8/56.9 | 40 | 11 | ESI(−) |
| OA | OA-13C18 | 281.1/281.1 | 40 | 4 | ESI(−) |
| LGPC | LGPC-d9 | 520.3→104.0 | 40 | 23 | ESI(+) |

FIG. 3A and FIG. 3B show the total ion flow chromatograms of standards of α-HB, OA, and LGPC and the total ion flow chromatograms of α-HB, OA, and LGPC in plasma, respectively. As shown, the peak shapes of α-HB, OA and LGPC in the standards and plasma samples were relatively symmetrical and without spurious peak interference, indicating that good detection could be obtained under these conditions.

The isotope internal standard quantification method was used to establish a calibration curve using TargetLynx™ software with a concentration ratio of standard to internal standard as the X-axis and a peak area ratio of standard to internal standard as the Y-axis. α-HB, OA and LGPC were linearly fitted to the equations in their respective concentration ranges with good linearity and correlation coefficients above 0.99, meeting the quantitative requirements, as shown in Table 10. According to the linear equations of the standard curve, the concentrations of the metabolites to be measured in plasma were calculated.

TABLE 10
Linear regression equations and linear correlation
coefficients of α-HB, OA and LGPC

| | Curve | | linear |
|---|---|---|---|
| Analytes | concentrations(μM) | Linear equations | coefficients (r) |
| α-HB | 2-200 | Y = 0.089415X − 0.472283 | 0.993 |
| OA | 10-1000 | Y = 0.020052X + 0.130601 | 0.998 |
| LGPC | 40-4000 | Y = 0.0486635X + 0.00615889 | 0.994 |

### Significance Tests for Metabolites in the GDM and Non-GDM Groups

The standard curves described above allowed the concentrations of individual metabolites to be determined, after which statistical analysis of significance was performed to identify significantly different metabolites. The statistical test of significance in the GDM and non-GDM groups was the Mann-Whitney U test, with a P value less than 0.05 being considered significant. The specific metabolites and their pathways and the P value results are shown in Table 11 below.

TABLE 11
Metabolite levels of subjects in the GDM and non-GDM groups

| | | | | | |
|---|---|---|---|---|---|
| | Total | | | | |
| | number | Non-GDM | GDM | | Biological |
| Variables | (n = 369) | (n = 241) | (n = 128) | P | pathways |

Glucose analogues

| | | | | | | | | |
|---|---|---|---|---|---|---|---|---|
| 1,5-AG* (μmol/L) | 51.10 | (32.43-77.78) | 58.98 | (41.13-83.77) | 38.12 | (23.86-60.56) | <0.001 | Glucose metabolism |

Organic acids

| | | | | | | | | |
|---|---|---|---|---|---|---|---|---|
| α-HB (μmol/L) | 34.33 | (27.12-43.86) | 31.16 | (23.82-38.59) | 42.11 | (33.79-50.99) | <0.001 | Methionine/threonine |
| | | | | | | | | metabolism |

Organic compounds

| | | | | | | | | |
|---|---|---|---|---|---|---|---|---|
| Ethanolamine (μmol/L) | 21.17 | (16.18-27.38) | 23.52 | (19.17-29.22) | 16.35 | (12.81-21.03) | <0.001 | Glycerophospholipid |
| | | | | | | | | metabolism |
| TMAO (μmol/L) | 1.68 | (1.11-2.57) | 1.72 | (1.18-2.50) | 1.63 | (0.94-2.78) | 0.468 | Intestinal |
| | | | | | | | | microbial metabolism |

Aromatic amino acids

| | | | | | | | | |
|---|---|---|---|---|---|---|---|---|
| L-Phenylalanine (μmol/L) | 54.88 | (49.53-63.03) | 56.69 | (51.80-64.95) | 51.31 | (46.63-58.82) | <0.001 | Phenylalanine |
| | | | | | | | | metabolism |
| L-Tryptophan (μmol/L) | 57.00 | (50.09-64.92) | 59.40 | (51.96-66.64) | 53.16 | (48.42-60.21) | <0.001 | Tryptophan |
| | | | | | | | | metabolism |
| L-Tyrosine (μmol/L) | 42.08 | (37.25-47.19) | 42.27 | (37.66-47.65) | 41.18 | (36.77-46.11) | 0.140 | Tyrosine |
| | | | | | | | | metabolism |

Branched-chain amino acids

| | | | | | | | | |
|---|---|---|---|---|---|---|---|---|
| L-Isoleucine (μmol/L) | 65.90 | (57.83-72.93) | 68.60 | (60.31-75.25) | 61.75 | (54.82-69.12) | <0.001 | Fatty acid oxidation, |
| L-Leucine (μmol/L) | 109.85 | (96.28-124.28) | 114.51 | (101.70-127.57) | 101.27 | (88.94-116.57) | <0.001 | mammalian rapamycin |
| L-Valine (μmol/L) | 179.67 | (163.01-202.36) | 182.12 | (164.95-202.36) | 173.29 | (157.03-201.55) | 0.111 | target protein, c- |
| | | | | | | | | Jun amino-terminal |
| | | | | | | | | kinase and insulin |
| | | | | | | | | receptor substrate |
| | | | | | | | | pathways |

Other amino acids

| | | | | | | | | |
|---|---|---|---|---|---|---|---|---|
| Citrulline (μmol/L) | 15.32 | (13.24-17.66) | 15.38 | (13.29-17.59) | 15.24 | (13.23-17.78) | 0.887 | Nitric oxide |
| | | | | | | | | biosynthesis |
| Cystine (μmol/L) | 10.45 | (8.28-12.63) | 9.98 | (7.92-11.42) | 11.86 | (9.46-14.77) | <0.001 | Amino acid |
| | | | | | | | | metabolism |
| Glutamine (μmol/L) | 324.80 | (282.07-366.62) | 332.42 | (286.67-364.88) | 317.06 | (266.71-366.90) | 0.156 | Amino acid |
| | | | | | | | | metabolism |
| Glutamic acid (μmol/L) | 104.28 | (77.55-147.08) | 108.05 | (86.98-147.08) | 88.90 | (64.21-143.41) | <0.001 | Glutamic acid |
| | | | | | | | | metabolism |
| Hydroxylysine * (μmol/L) | 0.475 | (0.382-0.595) | 0.463 | (0.374-0.555) | 0.524 | (0.404-0.661) | 0.001 | Amino acid |
| | | | | | | | | metabolism |
| L-Aspartic Acid (μmol/L) | 26.48 | (19.31-38.34) | 28.61 | (22.01-38.55) | 20.57 | (14.30-35.51) | 0.011 | Aspartic acid |
| | | | | | | | | metabolism |
| L-Alanine (μmol/L) | 315.20 | (270.24-367.07) | 328.26 | (290.44-376.10) | 288.63 | (244.80-335.40) | <0.001 | Glucose-alanine |
| | | | | | | | | cycle, glutamate, |
| | | | | | | | | glycine and |
| | | | | | | | | serine metabolism |
| L-Proline (μmol/L) | 121.10 | (102.99-142.00) | 121.41 | (104.48-141.67) | 119.92 | (100.25-143.28) | 0.718 | Amino acid |
| | | | | | | | | metabolism |
| L-Threonine (μmol/L) | 175.09 | (151.60-199.36) | 181.01 | (158.69-199.92) | 164.43 | (139.51-186.45) | <0.001 | Threonine |
| | | | | | | | | metabolism |
| Lysine (μmol/L) | 164.91 | (145.16-186.11) | 168.70 | (151.26-191.31) | 157.77 | (132.49-173.17) | <0.001 | Amino acid |
| | | | | | | | | metabolism |
| Methionine (μmol/L) | 22.85 | (20.18-26.09) | 23.94 | (20.87-26.76) | 21.20 | (19.37-23.78) | <0.001 | Amino acid |
| | | | | | | | | metabolism |
| Taurine (μmol/L) | 187.51 | (133.90-248.25) | 197.75 | (149.40-248.25) | 155.83 | (111.23-246.52) | <0.001 | Amino acid |
| | | | | | | | | metabolism |

Metabolic by-products

| | | | | | | | | |
|---|---|---|---|---|---|---|---|---|
| ADMA* (μmol/L) | 0.386 | (0.332-0.448) | 0.407 | (0.357-0.478) | 0.350 | (0.298-0.401) | <0.001 | ADMA |
| | | | | | | | | degradation |
| SDMA* (μmol/L) | 0.397 | (0.347-0.454) | 0.402 | (0.357-0.458) | 0.378 | (0.326-0.452) | 0.016 | Pro-inflammatory |
| | | | | | | | | effect |

Oleic acid

| | | | | | | | | |
|---|---|---|---|---|---|---|---|---|
| OA (μmol/L) | 136.24 | (101.91-165.63) | 129.16 | (95.79-155.31) | 151.41 | (121.15-176.96) | <0.001 | Fatty acid |
| | | | | | | | | metabolism |

Glycerophosphorylcholine

| | | | | | | | | |
|---|---|---|---|---|---|---|---|---|
| LPGC (μmol/L) | 8.86 | (7.33-10.77) | 9.00 | (7.72-10.68) | 8.51 | (6.57-10.84) | 0.087 | Glycerophospholipid |
| | | | | | | | | metabolism |

According to Table 11, it can be seen that the levels of cystine, hydroxylysine, α-HB and oleic acid were significantly higher in the GDM group compared to the non-GDM group (p<0.001); while 1,5-AG, ethanolamine, L-phenylalanine, L-tryptophan, L-isoleucine, L-leucine, L-aspartic acid, L-alanine, L-threonine, lysine, methionine, taurine asymmetric dimethylarginine, symmetric dimethylarginine and glutamic acid were significantly reduced (all p<0.01).

### Determination of the Prediction Model

### Model Acquisition Overview

The prediction model used in this embodiment is a logistic regression model, which is applicable to dichotomous problems. The model can be used to predict whether a subject is GDM.

The logistic regression model is a generalized linear model, assuming that variable y obeys a binomial distribution, the fitted form of the linear model is shown in equation (5) below:

$$log ( \frac{p}{1 - p} ) = {β}_{0} + {Σ}_{i = 1} {β}_{{i}^{x} i} \qquad ( 5 )$$

where p is the probability value that the subject is GDM,

$$log ( \frac{p}{1 - p} )$$

is the adds ratio, β₀ is the intercept, xᵢ is the various variables (e.g., various markers, age, pre-pregnancy BMI, etc.), and βᵢ is the slope.

The metabolite concentration data, age, pre-pregnancy BMI, and categorical information (i.e., whether the subjects were GDM) of 369 subjects were used as the sample data set. The above sample data set was divided into a training set and a validation set using a 10 times *10 fold cross validation method. The training and validation sets are used to estimate the β₀ and βᵢ parameters in Equation (5). Specifically, the optimal β₀ and βᵢ parameters are first evaluated based on the training set which provides variable data xᵢ and sample classification information, combined with the maximum probability estimation method. By determining β₀ and βᵢ, the trained model is obtained (i.e., the prediction model). Based on the data in the validation set and the trained model, the subjects in the validation set may be predicted, and the prediction results are compared with the true classification information. Finally, based on the computed results of the training and validation sets, the ROC curves are plotted and the AUC (Area Under the Curve of ROC) values of the ROC curves as well as the odds ratio and significance p-values of the variables in the model are calculated. The significance test for the variables in the logistic regression model was performed using the Wald test with a statistical significance criterion of P<0.05.

### Significance Tests for Variables in Each Prediction Model

Specifically, the age and pre-pregnancy BMI were risk factors known to be significantly related to the occurrence of GDM (p<0.001 in Table 1) and needed to be included as correction factors in all multivariate models. A prediction model only relating to age and pre-pregnancy BMI was designated as prediction model 1 and served as a control. The other metabolites were categorized according to their properties (see Table 11) and included in the models, respectively, and the ROC curves, AUC values, odds ratios, and significant P values for each variable in the multivariate models were analyzed according to the description of the above steps.

Based on the results of the above data, suitable multivariate models were screened based on a screening principle. The screening principle is that a screened model corresponds to the highest AUC value among the models relating to the same variables and the odds ratios of the variables in the screened model is statistically significant (statistical significance criterion P<0.05). The final screened multivariate models that met the screening principle were named: prediction model 2, prediction model 3, prediction model 4, and prediction model 5. The odds ratios of each variable in these five prediction models are shown in Table 12 below.

TABLE 12
Variables included in the five models as well
as p-values and odds ratios of each variable

| Model | Variable | P value | Odds ratio(95% CI) |
|---|---|---|---|
| Prediction | (Intercept in the | 1.229e−09 *** | 0.001(0.000, 0.011) |
| model1 | model equation) | | |
| | Age | 1.695e−03 ** | 1.084(1.031, 1.141) |
| | Pre-pregnancy BMI | 4.697e−05 *** | 1.163(1.083, 1.252) |
| Prediction | (Intercept in the | 7.149e−14 *** | 0.000(0.000, 0.000) |
| model 2 | model equation) | | |
| | α-HB | 7.635e−08 *** | 8.700(4.057, 19.728) |
| | Age | 4.413e−03 ** | 1.080(1.025, 1.139) |
| | Pre-pregnancy BMI | 2.918e−03 ** | 1.124(1.042, 1.216) |
| Prediction | (Intercept in the | 2.190e−02 * | 0.028(0.001, 0.581) |
| model 3 | model equation) | | |
| | 1,5-AG | 7.106e−07 *** | 0.341(0.220, 0.516) |
| | ADMA | 3.552e−04 *** | 0.132(0.041, 0.378) |
| | Age | 5.986e−03 ** | 1.080(1.023, 1.142) |
| | Pre-pregnancy BMI | 2.570e−03 ** | 1.128(1.044, 1.222) |
| Prediction | (Intercept in the | 1.667e−01 | 0.001(0.000, 17.625) |
| model 4 | model equation) | | |
| | Cystine | 1.376e−04 *** | 9.573(3.107, 31.978) |
| | Ethanolamine | 2.523e−11 *** | 0.001(0.000, 0.004) |
| | L-Leucine | 3.641e−02 * | 10.711(1.191, 103.409) |
| | L-Tryptophan | 9.444e−03 ** | 0.074(0.010, 0.520) |
| | Hydroxylysine | 3.554e−02 * | 2.873(1.083, 7.803) |
| | Taurine | 1.226e−05 *** | 35.338(7.799, 193.713) |
| | Age | 8.304e−03 ** | 1.092(1.024, 1.168) |
| | Pre-pregnancy BMI | 1.138e−02 * | 1.138(1.030, 1.259) |
| Prediction | (Intercept in the | 4.720e−02 * | 0.002(0.000, 0.857) |
| model 5 | model equation) | | |
| | 1,5-AG | 1.730e−05 *** | 0.308(0.176, 0.519) |
| | α-HB | 8.378e−05 *** | 7.900(2.939, 23.226) |
| | Taurine | 9.854e−03 ** | 8.842(1.772, 49.528) |
| | L-Aspartate | 2.477e−02 * | 3.995(1.234, 13.882) |
| | Cystine | 3.439e−03 ** | 6.219(1.895, 22.147) |
| | Ethanolamine | 1.118e−10 *** | 0.001(0.000, 0.005) |
| | Age | 2.627e−02 * | 1.082(1.010, 1.161) |
| | Pre-pregnancy BMI | 2.053e−02 * | 1.135(1.019, 1.266) |

where P-value * indicates significant, P-value ** indicates very significant, P-value *** indicates highly significant, and CI indicates confidence interval.

According to Table 12, it can be seen that the odds ratios of all variables of these five models screened were significant and all were in accordance with the screening principle. The age and pre-pregnancy BMI (both p<0.01) were significant in all five prediction models. The variables of the prediction model 2 included conventional risk factors (i.e., age and pre-pregnancy BMI) and α-HB (p<0.001). The variables of the prediction model 3 included the conventional risk factors, 1,5-AG, and ADMA (all p<0.001). The predictive model 4 included the conventional risk factors and amino acids, including cystine, ethanolamine, taurine, L-leucine, L-tryptophan and hydroxylysine (all p<0.05). The prediction model 5 included the conventional risk factors, α-HB, 1,5-AG, cystine, ethanolamine, taurine, and L-aspartate (all p<0.05). Using these multivariate models, levels of α-HB, 1,5-AG, ADMA, cystine, ethanolamine, taurine, leucine, tryptophan, L-aspartate, and hydroxylysine were significantly related to the occurrence of GDM.

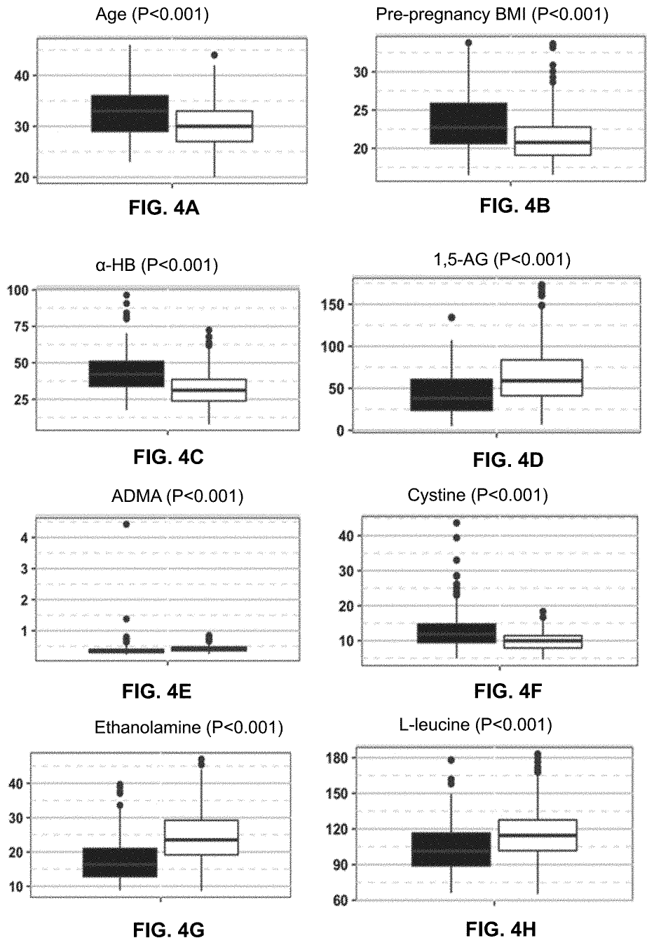

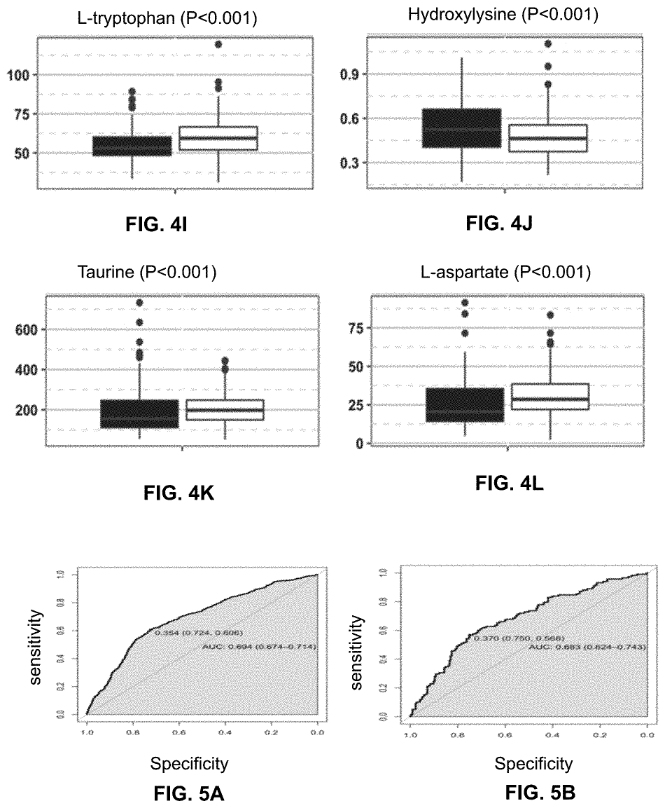

FIGS. 4A to 4L are distribution diagrams of the significant relationships of all five prediction models with GDM. The data distributions of the 12 variables involved in the 5 prediction models in the GDM and non-GDM groups are shown in FIG. 4A to FIG. 4L, from which it is clear that all these variables are significantly related to GDM.

### Determination of Prediction Model Parameters

According to equation (5), the variables xᵢ were entered for different models. The variables of the prediction model 1 were age and pre-pregnancy BMI, the variables of the prediction model 2 were age, pre-pregnancy BMI and α-HB, the variables of the prediction model 3 were age, pre-pregnancy BMI, 1,5-AG, ADMA, the variables of the prediction model 4 were age, pre-pregnancy BMI, cystine, ethanolamine, taurine, L-leucine L-tryptophan, and hydroxylysine, and the variables for prediction model 5 were age, pre-pregnancy BMI, α-HB, 1,5-AG, cystine, ethanolamine, taurine, and L-aspartate.

Based on the above variables and the real group data of subjects in the training set, the optimal values of the β₀ and βᵢ parameters in the five models were evaluated by the maximum probability estimation method to obtain each trained model (i.e., prediction models). The five prediction models are shown in Table 13 below.

TABLE 13
Equations of the 5 prediction models

| Prediction | |
|---|---|
| model | Equation |
| 1 | $\begin{matrix}log ( \frac{p}{1 - p} ) = - 6.52065 + 0.0 \; 8076 * age + 0.15063 * \\ pre ‐ pregnancy BMI\end{matrix}$ |
| 2 | $\begin{matrix}log ( \frac{p}{1 - p} ) = - 13.38647 + 1.4995 * ( α - HB ) + \\ 0.07 6 \; 65 * age + 0.11713 * \\ Pre ‐ pregnancy BMI\end{matrix}$ |
| 3 | $\begin{matrix}log ( \frac{p}{1 - p} ) = - 3.5 \; 6131 + ( - 0.74606 ) * ( 1 , 5 - A G ) + \\ ( - 1. 405 \; 08 ) * ADMA + 0.07688 * age + \\ 0.12063 * Pre ‐ pregnancy BMI\end{matrix}$ |
| 4 | $\begin{matrix}log ( \frac{p}{1 - p} ) = - 6.9 \; 8386 + 1.5 \; 6579 * Cystine + \\ ( - 5.25949 ) * Ethanolamine + 1.64365 * \\ ( L - Leucine ) + ( - 1. 806 \; 19 ) * ( L - \\ Tryptophan ) + 0.7315 * Hydroxylysine + \\ 2.47105 * Taurine + 0.08815 * age + \\ 0.12894 * Pre ‐ pregnancy BMI\end{matrix}$ |
| 5 | $\begin{matrix}log ( \frac{p}{1 - p} ) = - 6.3 \; 3027 + ( - 0.81716 ) * ( 1 , 5 - AG ) + \\ 1.43266 * ( α - HB ) + 1.51073 * Taurine + \\ 0.9601 * ( L - Aspartate ) + 1.26682 * \\ Cystine + ( - 5.1819 ) * Ethanolamine + \\ 0.0787 * Age + 0.127 * Pre ‐ pregnancy \\ BMI\end{matrix}$ |

### Calculation of Sensitivities, Specificities, Positive Predictive Values (PPV), and Negative Predictive Values (NPV) of Prediction Models

The 369 sample data were inputted into the equations of each prediction model in Table 13 above to calculate the sensitivity, specificity, PPV and NPV of each prediction model. The prediction model 1 is illustrated as an example. Based on the age and pre-pregnancy BMI of each sample and the equation of the prediction model 1, the probability value p of each sample belonging to the GDM group can be calculated. The probability value is within a range of [0,1], and values between [0,1] are divided into 201 quartiles (0th quantile is 0.0th, 1st quantile is 0.5th, 2nd quantile is 1.0th, 3rd quantile is 1.5th, 4th quantile is 2.0th, . . . , the 200th quantile 100th), each quantile corresponds to a value, which is referred to as a threshold. For the p-value of the first sample, if the p-value is greater than or equal to the threshold corresponding to the 0th quantile, the first sample is predicted to have GDM; if it is less than the threshold, the first sample is predicted to have non-GDM. Similarly, for the second sample to the 369th sample, the p-value of each sample was compared to the threshold corresponding to the 0th quantile to predict whether each sample is GDM. The samples with predicted diagnosis of GDM and non-GDM were compared with the true categories, and thus sensitivity, specificity, positive predictive value, and negative predictive value were calculated. Whether the samples are GDM or not can be predicted according to the threshold corresponding to the 0th quantile. the sensitivity, specificity, positive predictive value, and negative predictive value corresponding to each threshold were calculated. The sensitivity, specificity, positive predictive value, and negative predictive value of the remaining models were calculated in turn according to the above procedure.

Table 14 shows the comparison results of threshold ranges and the corresponding sensitivities, specificities, PPVs, and NPVs of the five prediction models. As shown in Table 14 below, there were not threshold ranges of the five prediction models under the condition that both sensitivities and specificities were greater than or equal to 85%, indicating none of them met this criterion (i.e., both sensitivity and specificity were greater than or equal to 85%). However, with sensitivities or specificities of 85%, the five models had the threshold ranges (data not shown).

With both sensitivity and specificity between [0.8, 0.85], a threshold range of [0.288597,0.323644] of the prediction model 5 was screened, i.e., any value within this threshold range can ensure that the sensitivity and specificity of the prediction model 5 are between [0.8, 0.85].

Under the condition that both sensitivities and specificities were between [0.75, 0.8], the prediction model 4 and the prediction model 5 had threshold ranges. The prediction model 5 had a wider threshold range, indicating that the prediction model 5 was more stable than the prediction model 4. Under the condition that the sensitivity, specificity, PPV and NPV were between [0.75, 0.8], only the prediction model 5 had the correlation threshold range.

With both sensitivities and specificities between [0.70, 0.75], the prediction model 3, the prediction model 4 and the prediction model 5 had correlation threshold ranges, wherein a threshold width of the prediction model 3 is less than a threshold width of the prediction model 4, and the threshold width of the prediction model 4 is less than a threshold width of the prediction model 5. With the sensitivities, specificities, PPVs and NPVs between [0.70, 0.75], the prediction model 4 and the prediction model 5 had the correlation threshold ranges while prediction model 3 did not have the correlation threshold range.

Under the condition that both sensitivities and specificities were between [0.65, 0.7], all five models had the threshold ranges with a threshold width of the prediction model 1<a threshold width of the prediction model 2<a threshold width of the prediction model 3<a threshold width of the prediction model 4<a threshold width of the prediction model 5. Under the condition that the sensitivities, specificities, PPVs and NPVs were between [0.65, 0.7], the prediction model 4 and prediction model 5 had the threshold ranges.

Under the condition that both sensitivities and specificities were between [0.60, 0.65], all five prediction models had the threshold ranges with a threshold width of the prediction model 1<a threshold width of the prediction model 2<a threshold width of the prediction model 3<a threshold width of the prediction model 4<a threshold width of the prediction model 5; under the condition that the sensitivities, specificities, PPVs and NPVs were between [0.60, 0.65], the prediction model 3, the prediction model 4 and the prediction model 5 had the threshold ranges with a threshold width of the prediction model 3<a threshold width of the prediction model 4<a threshold width of the prediction model 5.

TABLE 14
Comparison of the threshold ranges of the five prediction models

| | prediction model 1 | prediction model 2 | prediction model 3 | prediction model 4 | prediction model 5 |
|---|---|---|---|---|---|
| Both sensitivity and | | | | | |
| specificity are greater | | | | | |
| than or equal to 85% | | | | | |
| Sensitivity, specificity, | | | | | |
| PPV, NPV are greater | | | | | |
| than 85% | | | | | |
| Both sensitivity and | | | | | [0.288597, 0.323644] |
| specificity are greater | | | | | |
| than or equal to 80% | | | | | |
| Sensitivity, specificity, | | | | | |
| PPV, NPV are greater | | | | | |
| than 80% | | | | | |
| Both sensitivity and | | | | [0.274613, 0.323241] | [0.236272, 0.412465] |
| specificity are greater | | | | | |
| than or equal to 75% | | | | | |
| Sensitivity, specificity, | | | | | [0.384044, 0.412465] |
| PPV, NPV are greater | | | | | |
| than 75% | | | | | |
| Both sensitivity and | | | [0.317268, 0.360159] | [0.237638, 0.420441] | [0.198023, 0.546502] |
| specificity are greater | | | | | |
| than or equal to 70% | | | | | |
| Sensitivity, specificity, | | | | [0.333198, 0.420441] | [0.301805, 0.546502] |
| PPV, NPV are greater | | | | | |
| than 70% | | | | | |
| Both sensitivity and | [0.329666, 0.332614] | [0.309508, 0.374544] | [0.287868, 0.385842] | [0.207252, 0.466582] | [0.157141, 0.61763] |
| specificity are greater | | | | | |
| than or equal to 65% | | | | | |
| Sensitivity, specificity, | | | | [0.291602, 0.466582] | [0.23833, 0.61763] |
| PPV, NPV are greater | | | | | |
| than 65% | | | | | |
| Both sensitivity and | [0.313401, 0.356524] | [0.28913, 0.394162] | [0.257202, 0.415479] | [0.171792, 0.592092] | [0.132787, 0.66467] |
| specificity are greater | | | | | |
| than or equal to 60% | | | | | |
| Sensitivity, specificity, | | | [0.381516, 0.415479] | [0.240411, 0.592092] | [0.17302, 0.66467] |
| PPV, NPV are greater | | | | | |
| than 60% | | | | | |

The relationship between the threshold, sensitivity and specificity is that the larger the threshold, the higher the specificity, and the lower the sensitivity; the smaller the threshold, the higher the sensitivity, and the lower the specificity. The threshold range may be selected according to the sensitivity and specificity. For example, the sensitivity and specificity of the prediction model 5 are at [0.8, 0.85], and the threshold range [0.288597, 0.323644] corresponding to [0.8, 0.85] is selected. The sensitivity and specificity of the prediction model 4 are at, and the threshold range [0.274613, 0.323241] corresponding to [0.75, 0.8] is selected. The sensitivity and specificity of the prediction model 3 are at [0.7, 0.75], and the threshold range [0.317268, 0.360159] corresponding to [0.7, 0.75] is selected. The sensitivity and specificity of the prediction model 2 are at [0.65, 0.7], and the threshold range [0.309508, 0.374544] corresponding to [0.65, 0.7] is selected. The sensitivity and specificity of the prediction model 1 are at [0.65, 0.7], and the threshold range [0.329666, 0.332614] corresponding to [0.65, 0.7] is selected. The threshold of each prediction model may be chosen as needed from the threshold range.

### Evaluation of Each Prediction Model

ROC curves are drawn based on the sensitivity and specificity of each prediction model determined in the above steps. FIGS. 5A to 5J are ROC curves of five prediction models.

The evaluation data for the performance of the five prediction models according to FIG. 5A to FIG. 5J are shown in Table 15. The AUC of the prediction model 1 using the validation set was 0.683 (95% CI: 0.624-0.743). The AUC of the prediction model 2 using the validation set was 0.734 (95% CI: 0.679-0.789) with the addition of α-HB compared to the variables of the prediction model 1. The AUC of the prediction model 3 using the validation set was 0.773 with the addition of 1,5-AG and ADMA compared to the variables of the prediction model 1. The AUC of the prediction model 4 using the validation set was 0.852 (95% CI: 0.808-0.898) with the addition of cystine, ethanolamine, taurine, L-leucine, L-tryptophan and hydroxylysine compared to the variables of the prediction model 1. In particular, the AUC of the prediction model 5 using the validation set was 0.887 (0.849-0.926) with the addition of α-HB, 1,5-AG, cystine, ethanolamine, taurine, and L-aspartic acid compared to the variables of the prediction model 1. The higher AUC indicated the higher prediction accuracy of the prediction model. According to the AUCs of the five models from highest to lowest, the prediction model 5, the prediction model 4, the prediction model 3, the prediction model 2 and the prediction model 1 were ranked. Thus, the prediction models 2-5 can all be used to predict whether a subject has diabetes.

TABLE 15
AUCs of the training sets and AUCs of the validation
sets of the five prediction models

| | | Training set AUC | Validation set AUC |
|---|---|---|---|
| Model | Variable | (95% CI) | (95% CI) |
| Prediction | Age, pre-pregnancy | 0.694 (0.674-0.714) | 0.683 (0.624-0.743) |
| model 1 | BMI | | |
| Prediction | Age, pre-pregnancy | 0.745 (0.727-0.763) | 0.734 (0.679-0.789) |
| model 2 | BMI, α-HB | | |
| Prediction | Age, pre-pregnancy | 0.789 (0.771-0.806) | 0.773 (0.718-0.827) |
| model 3 | BMI, 1,5-AG, ADMA | | |
| Prediction | Age, pre-pregnancy | 0.877 (0.864-0.891) | 0.852 (0.808-0.898) |
| model 4 | BMI, cystine, | | |
| | ethanolamine, taurine, | | |
| | L-leucine, L-tryptophan | | |
| | and hydroxylysine | | |
| Prediction | Age, pre-pregnancy | 0.904 (0.893-0.915) | 0.887 (0.849-0.926) |
| model 5 | BMI, 1,5-AG, α-HB, | | |
| | cystine, ethanolamine, | | |
| | taurine, and L-aspartate | | |

According to FIG. 5A to FIG. 5J, considering only the values of the sensitivity and specificity, the threshold of each prediction model, as well as the corresponding sensitivity, specificity, positive predictive value, and negative predictive value may be determined by using the Jorden's index. Table 16 presents the results for the thresholds of the 5 prediction models and the corresponding sensitivities, specificities, positive predictive values, and negative predictive values.

TABLE 16
Results of sensitivities, specificities, positive predictive values and negative
predictive values of the five prediction models in the validation set

| Model | Sensitivity (%) | Specificity (%) | PPV (%) | NPV (%) | Threshold |
|---|---|---|---|---|---|
| Prediction | 56.8 | 75.0 | 54.5 | 76.7 | 0.370 |
| model 1 | | | | | |
| Prediction | 68.6 | 67.9 | 52.9 | 80.4 | 0.336 |
| model 2 | | | | | |
| Prediction | 72.0 | 71.9 | 57.4 | 83.0 | 0.336 |
| model 3 | | | | | |
| Prediction | 73.7 | 83.0 | 69.6 | 85.7 | 0.363 |
| model 4 | | | | | |
| Prediction | 74.6 | 87.5 | 75.9 | 86.7 | 0.413 |
| model 5 | | | | | |

It can be seen that the prediction model 5 was the best among the models according to the four indicators with a specificity of 87.5%, a sensitivity of 74.6%, a positive predictive value of 75.9%, a negative predictive value of 86.7%.

### Application of Prediction Models

For subjects with unknown classification of GDM, these 5 prediction models determined are used to predict whether the subjects are GDM.

First, a blood sample was taken from a subject, after which concentration values (e.g., in μmol/L) of the variables corresponding to the five prediction models were detected, and the subject's age and pre-pregnancy BMI values were obtained. These variables were input into the individual prediction models, and each prediction model output a probability value p. The probability value p was compared with a threshold corresponding to each prediction model (a threshold determined by the Jorden's index or selected from a threshold range), and if the probability value was greater than or equal to the threshold, the subject was predicted to have diabetes, e.g., GDM, type II diabetes; if the probability value was less than the threshold, the subject was predicted not to have diabetes, e.g., non-GDM, non-type II diabetes. The results of the five prediction models were compared to verify if the results were consistent. The prediction model 5 had the highest accuracy.

The results of the prediction models can provide an accurate reference to a physician for the subsequent diagnosis/treatment of a subject. For example, if a result of a prediction model is that a pregnant woman has GDM, OGTT testing can be used for further verification. Later, the physician can analyze the test results together with the clinical information of the pregnant woman, and can give further guidance on the future lifestyle of the pregnant woman or provide drug treatment.

The basic concepts have been described above, apparently, in detail, as described above, and does not constitute limitations of the disclosure. Although there is no clear explanation here, those skilled in the art may make various modifications, improvements, and modifications of present disclosure. This type of modification, improvement, and corrections are recommended in present disclosure, so the modification, improvement, and the amendment remain in the spirit and scope of the exemplary embodiment of the present disclosure.

At the same time, the present disclosure uses specific words to describe the embodiments of the present disclosure. As “one embodiment”, “an embodiment”, and/or “some embodiments” means a certain feature, structure, or characteristic of at least one embodiment of the present disclosure. Therefore, it is emphasized and should be appreciated that two or more references to “an embodiment”, “one embodiment” or “an alternative embodiment” in various parts of present disclosure are not necessarily all referring to the same embodiment. Further, certain features, structures, or features of one or more embodiments of the present disclosure may be combined.

In some embodiments, numbers expressing quantities of ingredients, properties, and so forth, configured to describe and claim certain embodiments of the application are to be understood as being modified in some instances by the term “about,” “approximate,” or “substantially”. Unless otherwise stated, “approximately”, “approximately” or “substantially” indicates that the number is allowed to vary by ±20%. Accordingly, in some embodiments, the numerical parameters used in the specification and claims are approximate values, and the approximate values may be changed according to characteristics required by individual embodiments. In some embodiments, the numerical parameters should be construed in light of the number of reported significant digits and by applying ordinary rounding techniques. Although the numerical domains and parameters used in the present disclosure are configured to confirm its range breadth, in the specific embodiment, the settings of such values are as accurately as possible within the feasible range.

For each patent, patent application, patent application publication and other materials referenced by the present disclosure, such as articles, books, instructions, publications, documentation, etc., hereby incorporated herein by reference. Except for the application history documents that are inconsistent with or conflict with the contents of the present disclosure, and the documents that limit the widest range of claims in the present disclosure (currently or later attached to the present disclosure). It should be noted that if a description, definition, and/or terms in the subsequent material of the present disclosure are inconsistent or conflicted with the content described in the present disclosure, the use of description, definition, and/or terms in this manual shall prevail.

Finally, it should be understood that the embodiments described herein are only configured to illustrate the principles of the embodiments of the present disclosure. Other deformations may also belong to the scope of the present disclosure. Thus, as an example, not limited, the alternative configuration of the present disclosure embodiment may be consistent with the teachings of the present disclosure. Accordingly, the embodiments of the present disclosure are not limited to the embodiments of the present disclosure clearly described and described.

1. A method for predicting a possibility of a subject with diabetes, comprising:
determining, based on a sample from the subject, a concentration of the marker, wherein the marker includes α-hydroxybutyric acid, 1,5-anhydroglucitol, cystine, ethanolamine, taurine, and L-aspartic acid; and
predicting, based on the concentration of the marker, the possibility of the subject with diabetes by using a prediction model related to the marker, the prediction model being further related to an age and body mass index (BMI) of the subject.

2. The method of claim 1, wherein the diabetes includes type 1 diabetes, type 2 diabetes, or gestational diabetes.

3-6. (canceled)

7. The method of claim 1, wherein the predicting, based on the concentration of the marker, the possibility of the subject with diabetes by using a prediction model related to the marker includes:
outputting a prediction value from the prediction model by using the concentration of the marker as an input to the prediction model; and
predicting the possibility of the subject having diabetes by comparing the prediction value to a threshold.

8. The method of claim 7, wherein the predicting the possibility of the subject having diabetes by comparing the prediction value to a threshold includes:
predicting that the possibility of the subject with diabetes is high if the prediction value is greater than or equal to the threshold; or
predicting that the possibility of the subject with diabetes is low if the prediction value is less than the threshold.

9. (canceled)

10. The method of claim 1, wherein the prediction model is represented by the equation of
$$log ( \frac{p}{1 - p} ) = - 13.38647 + 1.499 \; 50 * ( α - hydroxybutyric acid ) + 0.07665 * age + 0.11713 * BMI$$
where p represents a probability value of the subject with diabetes,
$$log ( \frac{p}{1 - p} )$$
represents an odds ratio, and α-hydroxybutyric acid represents a concentration of α-hydroxybutyric acid in μmol/L.

11. The method of claim 1, wherein the prediction model is represented by the equation of
$$log ( \frac{p}{1 - p} ) = - 3.56131 + ( - 0.74606 ) * ( 1 , 5 - anhydroglucitol ) + ( - 1.40508 ) * asymmetric dimethylarginine + 0.07688 * age + 0.12063 * BMI$$
where p represents a probability value of the subject with diabetes,
$$log ( \frac{p}{1 - p} )$$
represents an odds ratio, and 1,5-anhydroglucitol and asymmetric dimethylarginine represent a concentration of 1,5-anhydroglucitol and asymmetric dimethylarginine in μmol/L, respectively.

12. The method of claim 1, wherein the prediction model is represented by the equation of
$$log ( \frac{p}{1 - p} ) = - 6.98386 + 1.5 \; 6579 * cystine + ( 5.25949 ) * ethanolamine + 1.64365 * ( L - leucine ) + ( - 1.80619 ) * ( L - tryptophan ) + 0.7315 * hydroxylysine + 2.47105 * taurine + 0.08815 * age + 0.12894 * BMI$$
where p represents a probability value of the subject with diabetes,
$$log ( \frac{p}{1 - p} )$$
represents an odds ratio, and cystine, ethanolamine, L-leucine, L-tryptophan, hydroxylysine, and taurine represent concentrations of cystine, ethanolamine, L-leucine, L-tryptophan, hydroxylysine, and taurine in μmol/L, respectively.

13. The method of claim 1, wherein the prediction model is represented by the equation of
$$log ( \frac{p}{1 - p} ) = - 6.33027 + ( - 0.81716 ) * ( 1 , 5 - anhydroglucitol ) + 1.43266 ( α - hydroxybutyric acid ) + 1.51073 * taurine + 0.9601 * ( L - aspartic acid ) + 1.26682 * cystine + ( - 5.1819 ) * ethanolamine + 0.0787 * age + 0.127 * BMI$$
where p represents a probability value of the subject with diabetes,
$$log ( \frac{p}{1 - p} )$$
represents an odds ratio, 1,5-anhydroglucitol, α-hydroxybutyric acid, taurine, L-aspartic acid, cystine and ethanolamine represent concentrations of 1,5-anhydroglucitol, α-hydroxybutyric acid, taurine, L-aspartic acid, cystine and ethanolamine in μmol/L, respectively.

14. The method of claim 1, wherein all AUC values of the prediction model are greater than 0.7 in a validation set and a sensitivity and a specificity of the prediction model are greater than 65% in the validation set.

15-20. (canceled)

21. A method for predicting a possibility of a subject with diabetes by using a prediction model, wherein
the prediction model is related to a marker for predicting the possibility of the subject with diabetes, wherein the marker includes α-hydroxybutyric acid, 1,5-anhydroglucitol, cystine, ethanolamine, taurine, and L-aspartic acid;
an input of the prediction model is a concentration of the marker and an output of the prediction model is a prediction value, the prediction value is compared with a threshold to predict the possibility of the subject with diabetes, wherein the prediction model is a logistic regression model.

22. (canceled)

23. The method of claim 21, wherein the prediction model is further related to an age and BMI of the subject.

24. The method of claim 21, wherein all AUC values of the prediction model are greater than 0.7 in a validation set and a sensitivity and a specificity of the prediction model are greater than 65% in the validation set.

25-26. (canceled)
